(19) United States
(12) Patent Application Publication
(10) Pub. No.: US 2006/0040410 A1
(43) Pub. Date: Feb. 23, 2006

(54) Biosensor

(75) Inventors: Taisei Nishimi, Kanagawa (JP); Toshihide Ezoe, Kanagawa (JP); Toshiaki Kubo, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/205,183

(22) Filed: Aug. 17, 2005

(30) Foreign Application Priority Data
Aug. 18, 2004 (JP) 238396/2004
Sep. 22, 2004 (JP) 275012/2004

(51) Int. Cl. C12M 1/34; G01N 33/553

(52) U.S. Cl. 436/525; 435/287.2

(57) ABSTRACT

It is an object of the present invention to provide a technique of converting carboxylic acid into an active ester with no generation of air bubbles and a technique of stabilizing the obtained active ester. The present invention provides a biosensor wherein a carboxyl group existing on the surface of a substrate thereof is activated with any one compound selected from an uronium salt, a phosphonium salt or a triazine derivative which are defined in the present application, so as to form a carboxylic acid amide group.

### TECHNICAL FIELD

The present invention relates to a biosensor and a method for analyzing an interaction between biomolecules using the biosensor. Particularly, the present invention relates to a biosensor which is used for a surface plasmon resonance biosensor and a method for analyzing an interaction between biomolecules using the biosensor.

### BACKGROUND ART

Recently, a large number of measurements using intermolecular interactions such as immune responses are being carried out in clinical tests, etc. However, since conventional methods require complicated operations or labeling substances, several techniques are used that are capable of detecting the change in the binding amount of a test substance with high sensitivity without using such labeling substances. Examples of such a technique may include a surface plasmon resonance (SPR) measurement technique, a quartz crystal microbalance (QCM) measurement technique, and a measurement technique of using functional surfaces ranging from gold colloid particles to ultra-fine particles. The SPR measurement technique is a method of measuring changes in the refractive index near an organic functional film attached to the metal film of a chip by measuring a peak shift in the wavelength of reflected light, or changes in amounts of reflected light in a certain wavelength, so as to detect adsorption and desorption occurring near the surface. The QCM measurement technique is a technique of detecting adsorbed or desorbed mass at the ng level, using a change in frequency of a crystal due to adsorption or desorption of a substance on gold electrodes of a quartz crystal (device). In addition, the ultra-fine particle surface (nm level) of gold is functionalized, and physiologically active substances are immobilized thereon. Thus, a reaction to recognize specificity among physiologically active substances is carried out, thereby detecting a substance associated with a living organism from sedimentation of gold fine particles or sequences.

In all of the above-described techniques, the surface where a physiologically active substance is immobilized is important. Surface plasmon resonance (SPR), which is most commonly used in this technical field, will be described below as an example.

A commonly used measurement chip comprises a transparent substrate (e.g., glass), an evaporated metal film, and a thin film having thereon a functional group capable of immobilizing a physiologically active substance. The measurement chip immobilizes the physiologically active substance on the metal surface via the functional group. A specific binding reaction between the physiological active substance and a test substance is measured, so as to analyze an interaction between biomolecules.

As a thin film having a functional group capable of immobilizing a physiologically active substance, there has been reported a measurement chip where a physiologically active substance is immobilized by using a functional group binding to metal, a linker with a chain length of 10 or more atoms, and a compound having a functional group capable of binding to the physiologically active substance (Japanese Patent No. 2815120). Moreover, a measurement chip comprising a metal film and a plasma-polymerized film formed on the metal film has been reported (Japanese Patent Laid-Open No. 9-264843).

When the surface of the aforementioned measurement chip (biosensor) is produced, carboxylic acid amide is formed in water in many cases (a reaction of a polymer with a linker, and binding with a detected substance such as a protein). In order to conduct this reaction, 1-(3-dimethylaminopropyl)-3 ethylcarbodiimide (EDC) which is a water-soluble carbodiimide and N-hydroxysuccinimide (NHS) are generally used to activate carboxylic acid, and the activated carboxylic acid is then allowed to react with amine, so as to form carboxylic acid amide. In the case of producing a biosensor surface used in surface plasmon resonance analysis (SPR) or quartz crystal microbalance (QCM) as well, formation of an amide bond in water by the combined use of EDC with NHS has been reported (Japanese Patent Laid-Open No. 11-281569 and Japanese Patent Laid-Open No. 2000-39401). Likewise, production of an SPR surface has been disclosed in http://www.biacore.co.jp/2_{—}2_{—}1.shtml#a (Biacore), and production of a QCM surface has been disclosed in http://www.initium2000.com/technology.html (Initium).

However, when EDC is mixed with NHS in water, it has been problematic in that “air bubbles are generated as a result of the reaction.” In addition, it has also been problematic in that “the stability of the obtained active ester is not sufficient and it is hydrolyzed over time.” The former problem causes such a problem as “remaining of air bubbles in a flow channel,” when the aforementioned surface is applied to an SPR sensor wherein a reaction is required to be carried out in a hermetically closed narrow flow channel. The latter problem causes “a decrease in a reaction yield,” and thus an excessive amount of activator must be used with respect to carboxylic acid.

### DISCLOSURE OF THE INVENTION

It is an object of the present invention to solve the aforementioned problems of the prior art techniques. In other words, it is an object of the present invention to provide a technique of converting carboxylic acid into an active ester with no generation of air bubbles and a technique of stabilizing the obtained active ester.

As a result of intensive studies directed towards achieving the aforementioned object, the present inventors have found that carboxylic acid can be converted into an active ester with no generation of air bubbles, and the obtained active ester can be stabilized, by activating a carboxyl group existing on the surface of a substrate using any one compound selected from the group consisting of an uronium salt represented by the formula A1, a phosphonium salt represented by the formula A2, and a triazine derivative represented by the formula A3 which are defined in the present specification. Further, the present inventors have found that an active ester can be stabilized by activating a carboxyl group existing on the surface of a substrate with a carbodiimide derivative or a salt thereof, and converting it into an ester with any one compound selected from the group consisting of a nitrogen-containing heteroaromatic compound having a hydroxyl group, a phenol derivative having an electron-withdrawing group, and an aromatic compound having a thiol group. The present invention has been completed based in these findings.

Thus, the present invention provides a biosensor, wherein a carboxyl group existing on the surface of a substrate thereof is activated with any one compound selected from the group consisting of an uronium salt represented by the following formula A1, a phosphonium salt represented by the following formula A2, and a triazine derivative represented by the following formula A3, so as to form a carboxylic acid amide group: 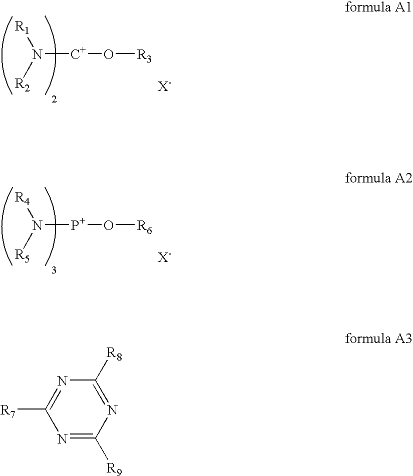
wherein, in the formula A1, each of R₁ and R₂ independently represents an alkyl group having 1 to 6 carbon atoms, or R₁ and R₂ together form an alkylene group having 2 to 6 carbon atoms, which forms a ring together with an N atom, R₃ represents an aromatic group having 6 to 20 carbon atoms or a heterocyclic group containing at least one heteroatom, and X⁻ represents an anion; in the formula A2, each of R₄ and R₅ independently represents an alkyl group having 1 to 6 carbon atoms, or R₄ and R₅ together form an alkylene group having 2 to 6 carbon atoms, which forms a ring together with an N atom, R₆ represents an aromatic group having 6 to 20 carbon atoms or a heterocyclic group containing at least one heteroatom, and X⁻ represents an anion; and in the formula A3, R₇ represents an onium group, and each of R₈ and R₉ independently represents an electron-donating group.

Preferably, the uronium salt represented by the formula A1 is any one compound selected from the following compounds A1 to A10 wherein X⁻ represents an anion.

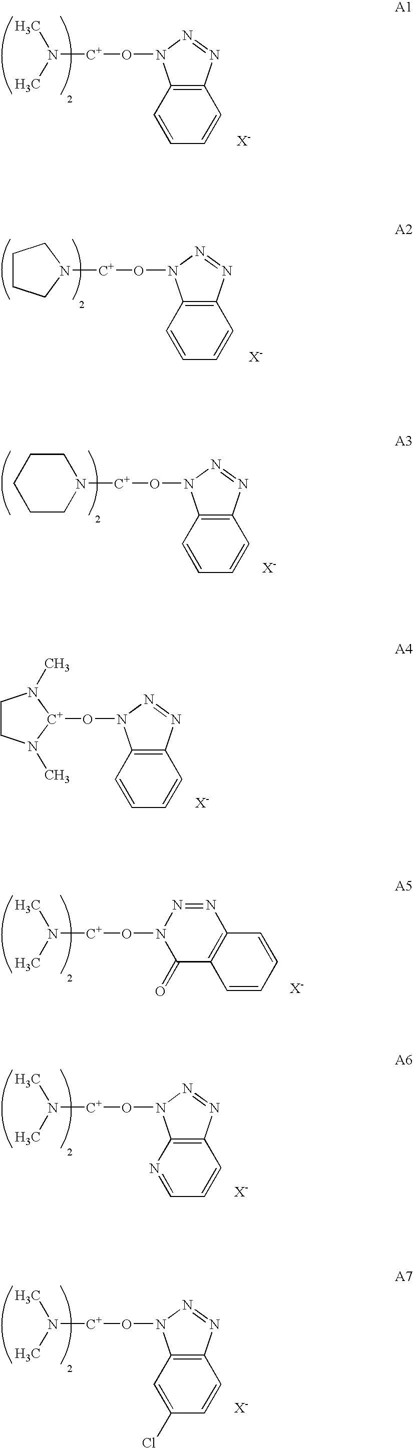

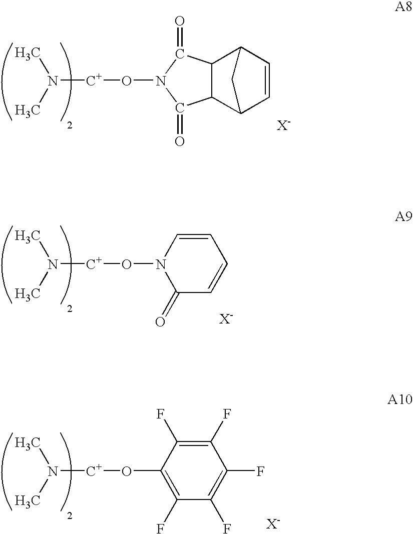

Preferably, X represents BF₄ or PF₆.

Preferably, the phosphonium salt represented by the formula 2 is any one compound selected from the following compounds A11 to A14 wherein X⁻ represents an anion.

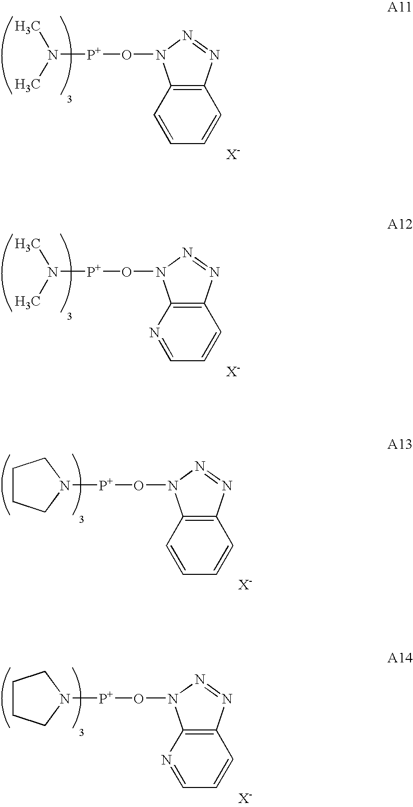

Preferably, X represents BF₄ or PF₆.

Preferably, the triazine derivative represented by the formula A3 is the following compound A15 wherein X⁻ represents an anion.

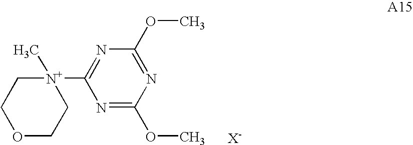

Preferably, X represents Cl.

Preferably, a substrate is coated with a hydrophobic polymer, and a carboxyl group contained in the above hydrophobic polymer is activated with the compound represented by any one of the formulas A1 to A3.

Another aspect of the present invention provides a biosensor, wherein a carboxyl group existing on the surface of a substrate thereof is activated with a carbodiimide derivative or a salt thereof, and it is then converted into an ester using any one compound selected from the group consisting of a nitrogen-containing heteroaromatic compound having a hydroxyl group, a phenol derivative having an electron-withdrawing group, and an aromatic compound having a thiol group, followed by a reaction with amine, so as to form a carboxylic acid amide group.

Preferably, the carbodiimide derivative or a salt thereof is water-soluble.

Preferably, the carbodiimide derivative is any one of the following compounds B1 to B3.

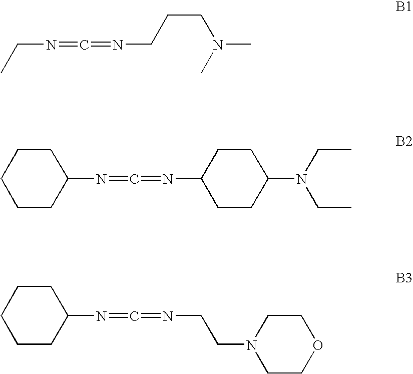

Preferably, the nitrogen-containing heteroaromatic compound having a hydroxyl group is any one of the following compounds B4 to B12.

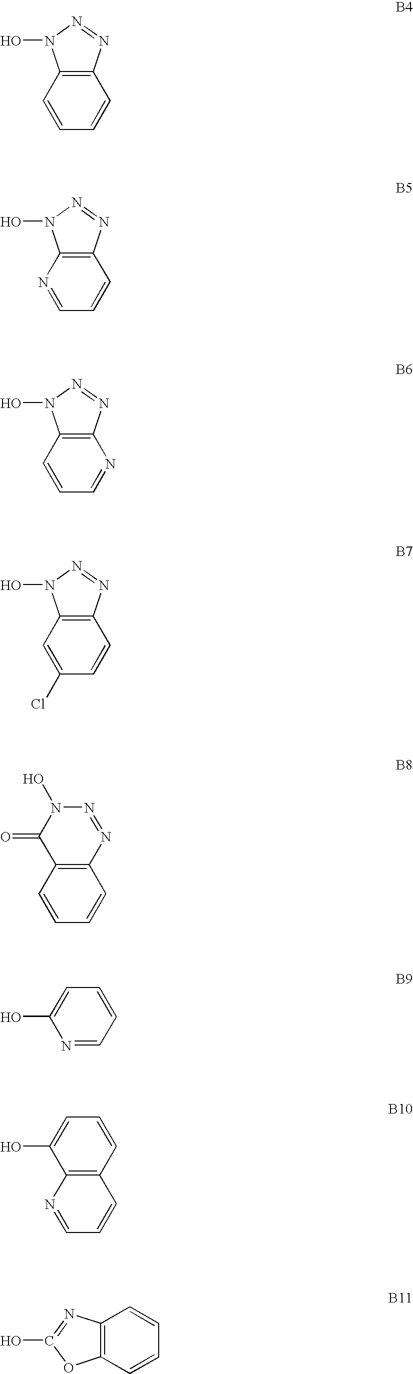

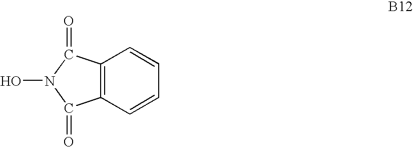

Preferably, in the phenol derivative having an electron-withdrawing group, the a value of the electron-withdrawing group is 0.3 or greater.

Preferably, the phenol derivative having an electron-withdrawing group is any one of the following compounds B13 to B16.

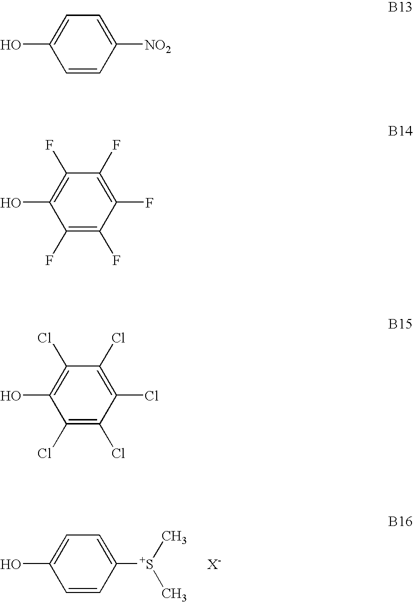

Preferably, the aromatic compound having a thiol group is any one of the following compounds B17 to B19.

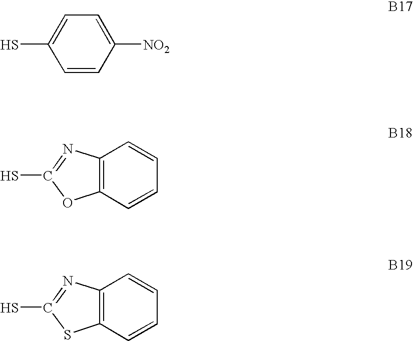

Preferably, a substrate is coated with a hydrophobic polymer, and a carboxyl group contained in said hydrophobic polymer is activated with a carbodiimide derivative or a salt thereof, and it is then converted into an ester with any one compound selected from the group consisting of a nitrogen-containing heteroaromatic compound having a hydroxyl group, a phenol derivative having an electron-withdrawing group, and an aromatic compound having a thiol group, followed by a reaction with amine, so as to form a carboxylic acid amide group.

Preferably, the coating thickness of the hydrophobic polymer is between 0.1 nm and 500 nm.

Preferably, the substrate is a metal surface or metal film.

Preferably, the metal surface or metal film consists of a free electron metal selected from the group consisting of gold, silver, copper, platinum, and aluminum.

Preferably, the biosensor of the present invention is used in non-electrochemical detection, and more preferably in surface plasmon resonance analysis.

Another aspect of the present invention provides a method for producing the aforementioned biosensor of the present invention, which comprises a step of activating a substrate having a carboxyl group on its surface with any one compound selected from the group consisting of an uronium salt represented by the following formula A1, a phosphonium salt represented by the following formula A2, and a triazine derivative represented by the following formula A3, so as to form a carboxylic acid amide group: 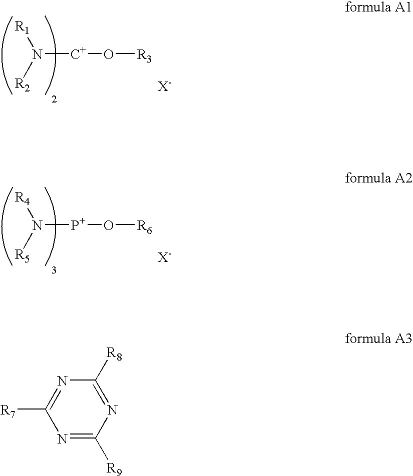
wherein, in the formula A1, each of R₁ and R₂ independently represents an alkyl group having 1 to 6 carbon atoms, or R₁ and R₂ together form an alkylene group having 2 to 6 carbon atoms, which forms a ring together with an N atom, R₃ represents an aromatic group having 6 to 20 carbon atoms or a heterocyclic group containing at least one heteroatom, and X⁻ represents an anion; in the formula A2, each of R₄ and R₅ independently represents an alkyl group having 1 to 6 carbon atoms, or R₄ and R₅ together form an alkylene group having 2 to 6 carbon atoms, which forms a ring together with an N atom, R₆ represents an aromatic group having 6 to 20 carbon atoms or a heterocyclic group containing at least one heteroatom, and X⁻ represents an anion; and in the formula A3, R₇ represents an onium group, and each of R₈ and R₉ independently represents an electron-donating group.

Further another aspect of the present invention provides a method for producing the biosensor of the present invention, which comprises steps of activating a substrate having a carboxyl group on its surface with a carbodiimide derivative or a salt thereof, and then converting it into an ester using any one compound selected from the group consisting of a nitrogen-containing heteroaromatic compound having a hydroxyl group, a phenol derivative having an electron-withdrawing group, and an aromatic compound having a thiol group, followed by a reaction with amine, so as to form a carboxylic acid amide group.

Further another aspect of the present invention provides the biosensor according to the present invention, wherein a physiologically active substance is bound to the surface by covalent bonding.

Further another aspect of the present invention provides a method for immobilizing a physiologically active substance on a biosensor, which comprises a step of allowing a physiologically active substance to come into contact with the biosensor according to the present invention, so as to allow said physiologically active substance to bind to the surface of said biosensor via a covalent bond.

Further another aspect of the present invention provides a method for detecting or measuring a substance interacting with a physiologically active substance, which comprises a step of allowing a test substance to come into contact with the biosensor according to the present invention to the surface of which the physiologically active substance binds via a covalent bond.

Preferably, the substance interacting with the physiologically active substance is detected or measured by a non-electrochemical method. More preferably, the substance interacting with the physiologically active substance is detected or measured by surface plasmon resonance analysis.

### BEST MODE FOR CARRYING OUT THE INVENTION

The embodiments of the present invention will be described below.

According to the first embodiment, the biosensor of the present invention is characterized in that a carboxyl group existing on the surface of a substrate thereof is activated with any one compound selected from the group consisting of an uronium salt represented by the following formula A1, a phosphonium salt represented by the following formula A2, and a triazine derivative represented by the following formula A3, so as to form a carboxylic acid amide group:

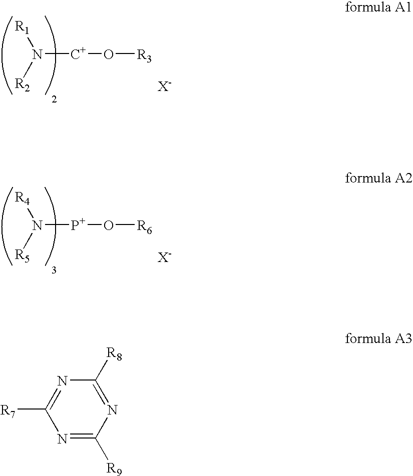

In the formula A1, each of R₁ and R₂ independently represents an alkyl group having 1 to 6 carbon atoms, or R₁ and R₂ together form an alkylene group having 2 to 6 carbon atoms, which forms a ring together with an N atom, R₃ represents an aromatic group having 6 to 20 carbon atoms or a heterocyclic group containing at least one heteroatom, and X⁻ represents an anion.

In the formula A2, each of R₄ and R₅ independently represents an alkyl group having 1 to 6 carbon atoms, or R₄ and R₅ together form an alkylene group having 2 to 6 carbon atoms, which forms a ring together with an N atom, R₆ represents an aromatic group having 6 to 20 carbon atoms or a heterocyclic group containing at least one heteroatom, and X⁻ represents an anion.

In the formula A3, R₇ represents an onium group, and each of R₈ and R₉ independently represents an electron-donating group.

Examples of an alkyl group having 1 to 6 carbon atoms may include a methyl group, an ethyl group, a propyl group, a butyl group, a pentyl group, and a hexyl group. These groups may have a linear or branched chain.

Examples of an alkylene group having 2 to 6 carbon atoms may include an ethylene group, a propylene group, a butylene group, a pentylene group, and hexylene group.

Examples of an aromatic group having 6 to 20 carbon atoms may include a phenyl group and a naphthyl group. It may also be possible that another ring is further condensed to these groups. In addition, such an aromatic group may have a substituent. Examples of a substituent may include a halogen atom (a fluorine atom, a chlorine atom, a bromine atom, or an iodine atom), an alkyl group, an alkenyl group, an alkynyl group, an aryl group, a heterocyclic group (including an N-substituted nitrogen-containing heterocyclic group, such as a morpholino group), an alkoxycarbonyl group, an aryloxycarbonyl group, a carbamoyl group, an N-substituted amide group, an imino group, an imino group substituted with an N atom, a thiocarbonyl group, a carbazoyl group, a cyano group, a thiocarbamoyl group, an alkoxy group, an aryloxy group, a heterocyclic oxy group, an acyloxy group, an (alkoxy or aryloxy)carbonyloxy group, a sulfonyloxy group, an acylamide group, a sulfonamide group, an ureide group, a thioureide group, an imide group, an (alkoxy or aryloxy)carbonylamino group, a sulfamoylamino group, a semicarbazide group, a thiosemicarbazide group, an (alkyl or aryl)sulfonylureide group, a nitro group, an (alkyl or aryl)sulfonyl group, a sulfamoyl group, a group having a phosphoric acid amide or phosphoric ester structure, a silyl group, a carboxyl group or a salt thereof, a sulfo group or a salt thereof, a phosphate group or a salt thereof, a hydroxy group, an ammonium group, a sulfonium group, a diazonium group, and an iodonium group.

A preferred example of a heterocyclic group containing at least one heteroatom may be a 5- or 7-membered saturated or unsaturated monocyclic or condensed ring containing one or more heteroatoms selected from the group consisting of a nitrogen atom, an oxygen atom, and a sulfur atom. Preferred examples of a heterocylic ring may include pyridine, quinoline, isoquinoline, pyrimidine, pyrazine, pyridazine, phthalazine, triazine, furan, thiophene, pyrrole, oxazole, benzoxazole, thiazole, benzothiazole, imidazole, benzoimidazole, thiadiazole, triazole, benzotriazole, 7-azabenzotriazole, and benzotriazine. Such a heterocylic group may have a substituent. Examples of such a substituent may be the same as the aforementioned substituents for an aromatic group.

Examples of an anion may include F⁻, Cl⁻, Br⁻, I⁻, At⁻, BF₄⁻, AsF₆⁻, PF₆⁻, SbF₆⁻, SbCl₆⁻, SnCl₆²⁻, FeCl₄^{-, BiCl}₅²⁻, CF₃SO₂⁻, ClO₄⁻, FSO₂⁻, and F₂PO₂⁻.

Examples of an onium group may include an ammonium group, a diazonium group, a piperidinium group, a morpholinium group, a quinuclidinium group, a pyridinium group, an anilinium group, a quinolinium group, an imidazolium group, an oxazolium group, a thiazolium group, an oxonium group, a sulfonium group, a selenonium group, a telluronium group, a phosphonium group, an arsonium group, a stibonium group, a bismuthonium group, a fluoronium group, a chloronium group, a bromonium group, an iodonium group, an oxonium group, a sulfonium group, a selenonium group, and a telluronium group.

Examples of an electron-donating group may include an alkyloxy group having 1 to 8 carbon atoms (for example, a methoxy group, an ethoxy group, a propyloxy group, a butyloxy group, a pentyloxy group, a hexyloxy group, a heptyloxy group, an octyloxy group, etc.), —NH₂, —OH, and —NR₂ (wherein R represents an alkyl group having 1 to 6 carbon atoms). Of these, an alkyloxy group having 1 to 8 carbon atoms is preferable, and a methoxy group is particularly preferable.

Specific examples of an uronium salt represented by the formula A1 may include any one compound selected from the following compounds A1 to A10 wherein X⁻ represents an anion, and preferably represents BF₄ or PF₆.

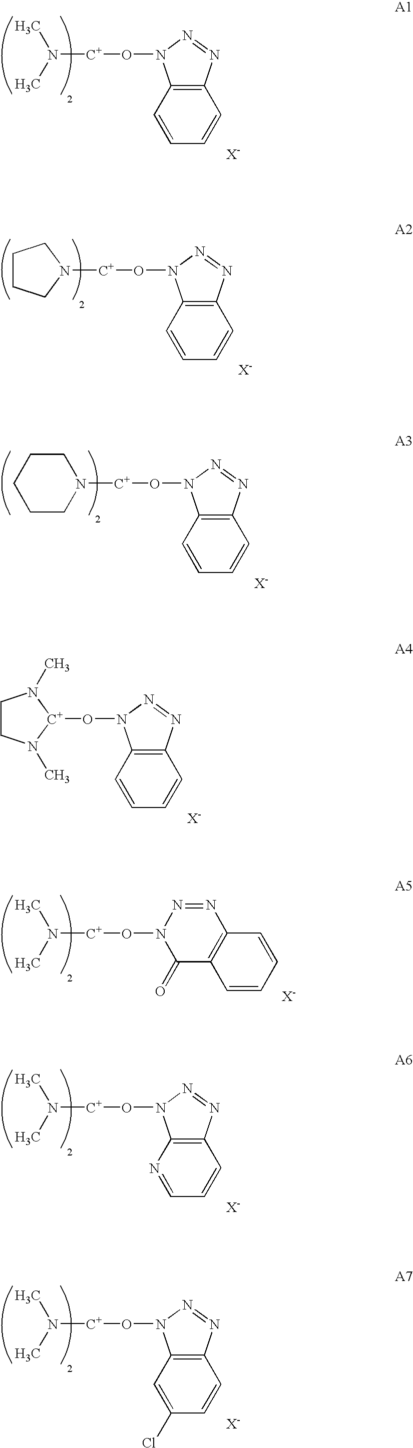

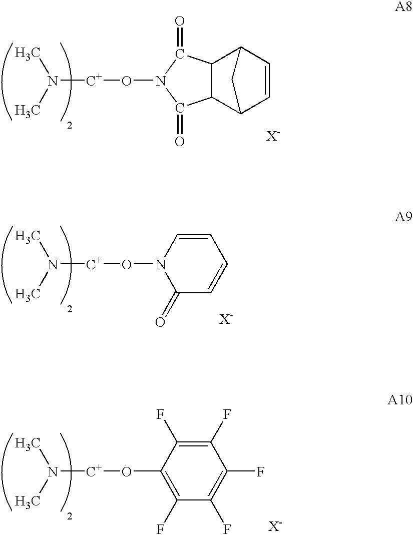

Specific examples of a phosphonium salt represented by the formula A2 may include any one compound selected from the following compounds A11 to A14 wherein X⁻ represents an anion, and preferably represents BF₄ or PF₆.

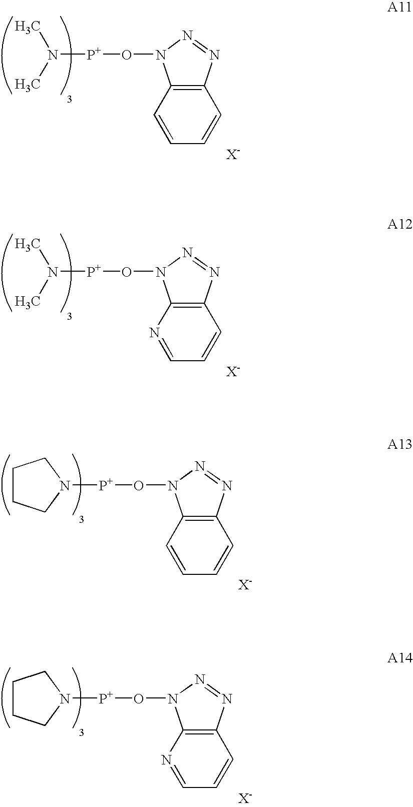

A specific example of a triazine derivative represented by the formula A3 may be the following compound A15 wherein X⁻ represents an anion, and preferably represents Cl.

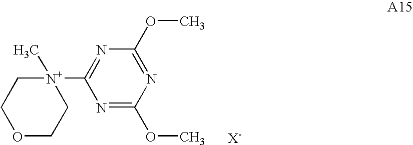

The compounds represented by the above-described formulas A1 to A3 (specific examples of which are compounds A1 to A15) are known compounds. These compounds can be synthesized by common methods, or commercially available products can be used as such compounds. Specifically, compounds A1 to A15 are commercially available from Kokusan Chemical, Aldrich, Sakai Kogyo, Dojindo Laboratories, etc. Otherwise, these compounds can be synthesized by methods described in the following publications (L. A. Carpino et al., J. Chem. Soc. Chem. Commun., 1994, 201., Y. Kiso et al., Chem. Pharm. Bull., 38, 270 (1990)).

A method for activating a carboxyl group existing on the surface of a substrate using the compounds represented by the above-described formulas A1 to A3 is not particularly limited, and common methods known to persons skilled in the art can be applied. Specifically, a solution containing the compounds represented by the above-described formulas A1 to A3 is come into contact with a substrate having a carboxyl group on the surface thereof, so as to activate the carboxyl group.

According to the second embodiment, the biosensor of the present invention is characterized in that a carboxyl group existing on the surface of a substrate thereof is activated with a carbodiimide derivative or a salt thereof, and it is then converted into an ester using any one compound selected from the group consisting of a nitrogen-containing heteroaromatic compound having a hydroxyl group, a phenol derivative having an electron-withdrawing group, and an aromatic compound having a thiol group, followed by a reaction with amine, so as to form a carboxylic acid amide group.

The carbodiimide derivative or a salt thereof is preferably water-soluble. Specifically, compounds B1 to B3 as described above in the present specification can be used, but examples are not limited thereto.

The type of a nitrogen-containing heteroaromatic compound having a hydroxyl group is not particularly limited. An example of such a nitrogen-containing heteroaromatic compound having a hydroxyl group may be a 5- or 7-membered saturated or unsaturated monocyclic or condensed ring containing at least one nitrogen atom (for example, 1, 2, or 3 nitrogen atoms). Specific examples of such a nitrogen-containing heteroaromatic compound having a hydroxyl group may include compounds B4 to B12 as described above in the present specification, but examples are not limited thereto.

Specific examples of an electron-withdrawing group contained in a phenol derivative having such an electron-withdrawing group may include —NO₂, halogen (—F, —Cl, —Br, or —I), —S(CH₃)₂X⁻ (wherein X represents a monovalent anion such as F⁻, Cl⁻, Br⁻, I⁻, At⁻, BF₄⁻, AsF₆⁻, PF₆⁻, SbF₆⁻, SbCl₆⁻, SnCl₆²⁻, FeCl₄⁻, BiCl₅²⁻, CF₃SO₂⁻, ClO₄⁻, FSO₂⁻, or F₂PO₂⁻), —COOH, —CN, and —CHO. The σ value of the electron-withdrawing group is preferably 0.3 or greater. The σ value of the electron-withdrawing group is more preferably 0.5 or greater, and further preferably 0.7 or greater. Such a value is summarized in the table included with Naoki Inamoto, “Hammette Soku (Hammette Measurement),” Maruzen (1983), for example. Specific examples of a phenol derivative having an electron-withdrawing group may include compounds B13 to B16 as described above in the present specification, but examples are not limited thereto.

The type of an aromatic compound having a thiol group (—SH) is not particularly limited. Examples of such an aromatic compound may include aryl compounds (benzene, naphthalene, etc.) and heterocyclic compounds containing at least one heteroatom. An example of a heterocyclic compound may be a 5- or 7-membered saturated or unsaturated monocyclic or condensed ring containing one or more heteroatoms selected from the group consisting of a nitrogen atom, an oxygen atom, and a sulfur atom. Specific examples may include pyridine, quinoline, isoquinoline, pyrimidine, pyrazine, pyridazine, phthalazine, triazine, furan, thiophene, pyrrole, oxazole, benzoxazole, thiazole, benzothiazole, imidazole, benzoimidazole, thiadiazole, triazole, benzotriazole, 7-azabenzotriazole, and benzotriazine. Specific examples of an aromatic compound having a thiol group may include compounds B17 to B19 as described above in the present specification, but examples are not limited thereto.

A method of activating a carboxyl group existing on the surface of a substrate with a carbodiimide derivative or a salt thereof, and then converting it into an ester with any one compound selected from the group consisting of a nitrogen-containing heteroaromatic compound having a hydroxyl group, a phenol derivative having an electron-withdrawing group, and an aromatic compound having a thiol group, is not particularly limited. Such operations can be carried out according to common methods known to persons skilled in the art. Specifically, a solution (for example, an aqueous solution) containing a carbodiimide derivative or a salt thereof and any compound selected from the group consisting of a nitrogen-containing heteroaromatic compound having a hydroxyl group, a phenol derivative having an electron-withdrawing group, and an aromatic compound having a thiol group, is come into contact with a substrate having a carboxyl group on the surface thereof, so as to esterify the above carboxyl group.

The aforementioned compounds B1 to B3 and B4 to B19 are known compounds, which can be synthesized by common methods. Otherwise, commercially available products can be used as such compounds. Specifically, compounds B1 to B19 are available from Kokusan Chemical, Aldrich, Sakai Kogyo, Dojindo Laboratories, etc. Alternatively, these compounds can be synthesized by methods described in the following publications (Bull. Chem. Soc. Jpn., 60, 2409 (1987)., J. Polym. Sci., Polym. Chem. Ed., 17 2013(1979)., J. Polym. Sci., Polym. Chem. Ed., 16, 475 (1978)).

In the biosensor of the present invention, the active ester formed as described above is allowed to react with amine, so as to form a carboxylic acid amide group. A method of reacting the active ester with amine is not particularly limited, and the reaction can be carried out by common methods known to persons skilled in the art. For example, the reaction may be carried out by allowing an ethanolamine-HCl solution to come into contact with a substrate.

The biosensor of the present invention has as broad a meaning as possible, and the term biosensor is used herein to mean a sensor, which converts an interaction between biomolecules into a signal such as an electric signal, so as to measure or detect a target substance. The conventional biosensor is comprised of a receptor site for recognizing a chemical substance as a detection target and a transducer site for converting a physical change or chemical change generated at the site into an electric signal. In a living body, there exist substances having an affinity with each other, such as enzyme/substrate, enzyme/coenzyme, antigen/antibody, or hormone/receptor. The biosensor operates on the principle that a substance having an affinity with another substance, as described above, is immobilized on a substrate to be used as a molecule-recognizing substance, so that the corresponding substance can be selectively measured.

In a preferred aspect of the present invention, a substrate is coated with a hydrophobic polymer, and a carboxyl group contained in the hydrophobic polymer can be activated with a compound as mentioned hereinabove.

A hydrophobic polymer used in the present invention is a polymer having no water-absorbing properties. Its solubility in water (25° C.) is 10% or less, more preferably 1% or less, and most preferably 0.1% or less.

A hydrophobic monomer which forms a hydrophobic polymer can be selected from vinyl esters, acrylic esters, methacrylic esters, olefins, styrenes, crotonic esters, itaconic diesters, maleic diesters, fumaric diesters, allyl compounds, vinyl ethers, vinyl ketones, or the like. The hydrophobic polymer may be either a homopolymer consisting of one type of monomer, or copolymer consisting of two or more types of monomers.

Examples of a hydrophobic polymer that is preferably used in the present invention may include polystyrene, polyethylene, polypropylene, polyethylene terephthalate, polyvinyl chloride, polymethyl methacrylate, polyester, and nylon.

A substrate is coated with a hydrophobic polymer according to common methods. Examples of such a coating method may include spin coating, air knife coating, bar coating, blade coating, slide coating, curtain coating, spray method, evaporation method, cast method, and dip method. Among them, spin coating and dip method are mentioned in detail below.

Spin-coating is a method for producing a thin film by adding a solution dropwise to a substrate placed on a rotating disk, wherein the thickness of a film is controlled by the concentration of the solution, the number of rotations of the disk, the vapor pressure of a solvent, etc.

The concentration of a hydrophobic polymer contained in a solution used in the spin-coating is preferably between 0.001% by weight and 50% by weight, more preferably between 0.01% by weight and 10% by weight, and further preferably between 0.1% by weight and 5% by weight.

The number of rotations of the disk during spin coating is preferably between 10 rpm and 10,000 rpm, more preferably between 50 rpm and 7,500 rpm, and further preferably between 100 rpm and 5,000 rpm.

A solvent used in the spin-coating has a vapor pressure preferably between 0.1 kPa and 100 kPa, more preferably between 0.5 kPa and 50 kPa, and further preferably between 1 kPa and 30 kPa, at the environmental temperature applied during the production of a thin film by spin coating. Specific examples of a solvent used herein may include methanol, ethanol, i-propanol, n-butanol, t-butanol, acetone, methyl ethyl ketone, methyl isobutyl ketone, ethyl acetate, butyl acetate, dimethyl sulfoxide, and dimethyl formamide.

A method for producing the biosensor of the present invention preferably comprises adding a coating solution dropwise to the surface of a substrate retained on a disk and then rotating the disk.

When the coating solution is added dropwise to the substrate surface, the coating rate on the substrate surface with the coating solution is preferably between 80% and 100%, and preferably between 90% and 100%.

In the dip method, coating is carried out by contacting a substrate with a solution of a hydrophobic polymer, and then with a liquid which does not contain the hydrophobic polymer. Preferably, the solvent of the solution of a hydrophobic polymer is the same as that of the liquid which does not contain said hydrophobic polymer.

In the dip method, a layer of a hydrophobic polymer having an uniform coating thickness can be obtained on a surface of a substrate regardless of inequalities, curvature and shape of the substrate by suitably selecting a coating solvent for hydrophobic polymer.

The type of coating solvent used in the dip method is not particularly limited, and any solvent can be used so long as it can dissolve a part of a hydrophobic polymer. Examples thereof include formamide solvents such as N,N-dimethylformamide, nitrile solvents such as acetonitrile, alcohol solvents such as phenoxyethanol, ketone solvents such as 2-butanone, and benzene solvents such as toluene, but are not limited thereto.

In the solution of a hydrophobic polymer which is contacted with a substrate, the hydrophobic polymer may be dissolved completely, or alternatively, the solution may be a suspension which contains undissolved component of the hydrophobic polymer. The temperature of the solution is not particularly limited, so long as the state of the solution allows a part of the hydrophobic polymer to be dissolved. The temperature is preferably −20° C. to 100° C. The temperature of the solution may be changed during the period when the substrate is contacted with a solution of a hydrophobic polymer. The concentration of the hydrophobic polymer in the solution is not particularly limited, and is preferably 0.01% to 30%, and more preferably 0.1% to 10%.

The period for contacting the solid substrate with a solution of a hydrophobic polymer is not particularly limited, and is preferably 1 second to 24 hours, and more preferably 3 seconds to 1 hour.

As the liquid which does not contain the hydrophobic polymer, it is preferred that the difference between the SP value (unit: (J/cm³)^{1/2}) of the solvent itself and the SP value of the hydrophobic polymer is 1 to 20, and more preferably 3 to 15. The SP value is represented by a square root of intermolecular cohesive energy density, and is referred to as solubility parameter. In the present invention, the SP value δ0 was calculated by the following formula. As the cohesive energy (Ecoh) of each functional group and the mol volume (V), those defined by Fedors were used (R. F. Fedors, Polym. Eng. Sci., 14(2), P 147, P 472 (1974)). Δ=(ΣEcoh/ΣV)^{1/2}

Examples of the SP values of the hydrophobic polymers and the solvents are shown below;
Solvent: 2-phenoxyethanol: 25.3 against polymethylmethacrylate-polystyrene copolymer (1:1): 21.0
Solvent: acetonitrile: 22.9 against polymethylmethacrylate: 20.3
Solvent: toluene: 18.7 against polystyrene: 21.6

The period for contacting a substrate with a liquid which does not contain the hydrophobic polymer is not particularly limited, and is preferably 1 second to 24 hours, and more preferably 3 seconds to 1 hour. The temperature of the liquid is not particularly limited, so long as the solvent is in a liquid state, and is preferably −20° C. to 100° C. The temperature of the liquid may be changed during the period when the substrate is contacted with the solvent. When a less volatile solvent is used, the less volatile solvent may be substituted with a volatile solvent which can be dissolved in each other after the substrate is contacted with the less volatile solvent, for the purpose of removing the less volatile solvent.

The coating thickness of a hydrophobic polymer is not particularly limited, but it is preferably between 0.1 nm and 500 nm, and particularly preferably between 1 nm and 300 nm.

Preferably, in the biosensor of the present invention, a metal surface or metal film is coated with a hydrophobic polymer. A metal constituting the metal surface or metal film is not particularly limited, as long as surface plasmon resonance is generated when the metal is used for a surface plasmon resonance biosensor. Examples of a preferred metal may include free-electron metals such as gold, silver, copper, aluminum or platinum. Of these, gold is particularly preferable. These metals can be used singly or in combination. Moreover, considering adherability to the above substrate, an interstitial layer consisting of chrome or the like may be provided between the substrate and a metal layer.

The film thickness of a metal film is not limited. When the metal film is used for a surface plasmon resonance biosensor, the thickness is preferably between 0.1 nm and 500 nm, and particularly preferably between 1 nm and 200 nm. If the thickness exceeds 500 nm, the surface plasmon phenomenon of a medium cannot be sufficiently detected. Moreover, when an interstitial layer consisting of chrome or the like is provided, the thickness of the interstitial layer is preferably between 0.1 nm and 10 nm.

Formation of a metal film may be carried out by common methods, and examples of such a method may include sputtering method, evaporation method, ion plating method, electroplating method, and nonelectrolytic plating method.

A metal film is preferably placed on a substrate. The description “placed on a substrate” is used herein to mean a case where a metal film is placed on a substrate such that it directly comes into contact with the substrate, as well as a case where a metal film is placed via another layer without directly coming into contact with the substrate. When a substrate used in the present invention is used for a surface plasmon resonance biosensor, examples of such a substrate may include, generally, optical glasses such as BK7, and synthetic resins. More specifically, materials transparent to laser beams, such as polymethyl methacrylate, polyethylene terephthalate, polycarbonate or a cycloolefin polymer, can be used. For such a substrate, materials that are not anisotropic with regard to polarized light and have excellent workability are preferably used.

The biosensor of the present invention comprising a substrate coated with a hydrophobic polymer preferably has a functional group capable of immobilizing a physiologically active substance on the outermost surface of the substrate. The term “the outermost surface of the substrate” is used to mean “the surface, which is farthest from the substrate,” and more specifically, it means “the surface of a hydrophobic polymer applied on a substrate, which is farthest from the substrate.”

In order to introduce these functional groups into the outermost surface, a method is applied that involves applying a hydrophobic polymer containing a precursor of such a functional group on a metal surface or metal film, and then generating the functional group from the precursor located on the outermost surface by chemical treatment. For example, polymethyl methacrylate, a hydrophobic polymer containing —COOCH₃ group is coated on a metal film, and then the surface comes into contact with an NaOH aqueous solution (1N) at 40° C. for 16 hours, so that a —COOH group is generated on the outermost surface.

In the surface of the biosensor obtained as mentioned above, the —COOH group is activated by the method of the present invention to form a carboxylic acid amide group, and then a physiologically active substance is covalently bound via the thus-activated carboxylic acid amide group, so that the physiologically active substance can be immobilized on the metal surface or metal film.

A physiologically active substance immobilized on the surface for the biosensor of the present invention is not particularly limited, as long as it interacts with a measurement target. Examples of such a substance may include an immune protein, an enzyme, a microorganism, nucleic acid, a low molecular weight organic compound, a nonimmune protein, an immunoglobulin-binding protein, a sugar-binding protein, a sugar chain recognizing sugar, fatty acid or fatty acid ester, and polypeptide or oligopeptide having a ligand-binding ability.

Examples of an immune protein may include an antibody whose antigen is a measurement target, and a hapten. Examples of such an antibody may include various immunoglobulins such as IgG, IgM, IgA, IgE or IgD. More specifically, when a measurement target is human serum albumin, an anti-human serum albumin antibody can be used as an antibody. When an antigen is an agricultural chemical, pesticide, methicillin-resistant Staphylococcus aureus, antibiotic, narcotic drug, cocaine, heroin, crack or the like, there can be used, for example, an anti-atrazine antibody, anti-kanamycin antibody, anti-metamphetamine antibody, or antibodies against O antigens 26, 86, 55, 111 and 157 among enteropathogenic Escherichia coli.

An enzyme used as a physiologically active substance herein is not particularly limited, as long as it exhibits an activity to a measurement target or substance metabolized from the measurement target. Various enzymes such as oxidoreductase, hydrolase, isomerase, lyase or synthetase can be used. More specifically, when a measurement target is glucose, glucose oxidase is used, and when a measurement target is cholesterol, cholesterol oxidase is used. Moreover, when a measurement target is an agricultural chemical, pesticide, methicillin-resistant Staphylococcus aureus, antibiotic, narcotic drug, cocaine, heroin, crack or the like, enzymes such as acetylcholine esterase, catecholamine esterase, noradrenalin esterase or dopamine esterase, which show a specific reaction with a substance metabolized from the above measurement target, can be used.

A microorganism used as a physiologically active substance herein is not particularly limited, and various microorganisms such as Escherichia coli can be used.

As nucleic acid, those complementarily hybridizing with nucleic acid as a measurement target can be used. Either DNA (including cDNA) or RNA can be used as nucleic acid. The type of DNA is not particularly limited, and any of native DNA, recombinant DNA produced by gene recombination and chemically synthesized DNA may be used.

As a low molecular weight organic compound, any given compound that can be synthesized by a common method of synthesizing an organic compound can be used.

A nonimmune protein used herein is not particularly limited, and examples of such a nonimmune protein may include avidin (streptoavidin), biotin, and a receptor.

Examples of an immunoglobulin-binding protein used herein may include protein A, protein G, and a rheumatoid factor (RF).

As a sugar-binding protein, for example, lectin is used.

Examples of fatty acid or fatty acid ester may include stearic acid, arachidic acid, behenic acid, ethyl stearate, ethyl arachidate, and ethyl behenate.

When a physiologically active substance is a protein such as an antibody or enzyme or nucleic acid, an amino group, thiol group or the like of the physiologically active substance is covalently bound to a functional group located on a metal surface, so that the physiologically active substance can be immobilized on the metal surface.

A biosensor to which a physiologically active substance is immobilized as described above can be used to detect and/or measure a substance which interacts with the physiologically active substance.

Thus, the present invention provides a method of detecting and/or measuring a substance interacting with the physiologically active substance immobilized to the biosensor of the present invention, to which a physiologically active substance is immobilized, wherein the biosensor is contacted with a test substance.

As such a test substance, for example, a sample containing the above substance interacting with the physiologically active substance can be used.

In the present invention, it is preferable to detect and/or measure an interaction between a physiologically active substance immobilized on the surface used for a biosensor and a test substance by a nonelectric chemical method. Examples of a non-electrochemical method may include a surface plasmon resonance (SPR) measurement technique, a quartz crystal microbalance (QCM) measurement technique, and a measurement technique that uses functional surfaces ranging from gold colloid particles to ultra-fine particles.

In a preferred embodiment of the present invention, the biosensor of the present invention can be used as a biosensor for surface plasmon resonance which is characterized in that it comprises a metal film placed on a transparent substrate.

A biosensor for surface plasmon resonance is a biosensor used for a surface plasmon resonance biosensor, meaning a member comprising a portion for transmitting and reflecting light emitted from the sensor and a portion for immobilizing a physiologically active substance. It may be fixed to the main body of the sensor or may be detachable.

The surface plasmon resonance phenomenon occurs due to the fact that the intensity of monochromatic light reflected from the border between an optically transparent substance such as glass and a metal thin film layer depends on the refractive index of a sample located on the outgoing side of the metal. Accordingly, the sample can be analyzed by measuring the intensity of reflected monochromatic light.

A device using a system known as the Kretschmann configuration is an example of a surface plasmon measurement device for analyzing the properties of a substance to be measured using a phenomenon whereby a surface plasmon is excited with a lightwave (for example, Japanese Patent Laid-Open No. 6-167443). The surface plasmon measurement device using the above system basically comprises a dielectric block formed in a prism state, a metal film that is formed on a face of the dielectric block and comes into contact with a measured substance such as a sample solution, a light source for generating a light beam, an optical system for allowing the above light beam to enter the dielectric block at various angles so that total reflection conditions can be obtained at the interface between the dielectric block and the metal film, and a light-detecting means for detecting the state of surface plasmon resonance, that is, the state of attenuated total reflection, by measuring the intensity of the light beam totally reflected at the above interface.

In order to achieve various incident angles as described above, a relatively thin light beam may be caused to enter the above interface while changing an incident angle. Otherwise, a relatively thick light beam may be caused to enter the above interface in a state of convergent light or divergent light, so that the light beam contains components that have entered therein at various angles. In the former case, the light beam whose reflection angle changes depending on the change of the incident angle of the entered light beam can be detected with a small photodetector moving in synchronization with the change of the above reflection angle, or it can also be detected with an area sensor extending along the direction in which the reflection angle is changed. In the latter case, the light beam can be detected with an area sensor extending to a direction capable of receiving all the light beams reflected at various reflection angles.

With regard to a surface plasmon measurement device with the above structure, if a light beam is allowed to enter the metal film at a specific incident angle greater than or equal to a total reflection angle, then an evanescent wave having an electric distribution appears in a measured substance that is in contact with the metal film, and a surface plasmon is excited by this evanescent wave at the interface between the metal film and the measured substance. When the wave vector of the evanescent light is the same as that of a surface plasmon and thus their wave numbers match, they are in a resonance state, and light energy transfers to the surface plasmon. Accordingly, the intensity of totally reflected light is sharply decreased at the interface between the dielectric block and the metal film. This decrease in light intensity is generally detected as a dark line by the above light-detecting means. The above resonance takes place only when the incident beam is p-polarized light. Accordingly, it is necessary to set the light beam in advance such that it enters as p-polarized light.

If the wave number of a surface plasmon is determined from an incident angle causing the attenuated total reflection (ATR), that is, an attenuated total reflection angle (θSP), the dielectric constant of a measured substance can be determined. As described in Japanese Patent Laid-Open No. 11-326194, a light-detecting means in the form of an array is considered to be used for the above type of surface plasmon measurement device in order to measure the attenuated total reflection angle (θSP) with high precision and in a large dynamic range. This light-detecting means comprises multiple photo acceptance units that are arranged in a certain direction, that is, a direction in which different photo acceptance units receive the components of light beams that are totally reflected at various reflection angles at the above interface.

In the above case, there is established a differentiating means for differentiating a photodetection signal outputted from each photo acceptance unit in the above array-form light-detecting means with regard to the direction in which the photo acceptance unit is arranged. An attenuated total reflection angle (θSP) is then specified based on the derivative value outputted from the differentiating means, so that properties associated with the refractive index of a measured substance are determined in many cases.

In addition, a leaking mode measurement device described in “Bunko Kenkyu (Spectral Studies)” Vol. 47, No. 1 (1998), pp. 21 to 23 and 26 to 27 has also been known as an example of measurement devices similar to the above-described device using attenuated total reflection (ATR). This leaking mode measurement device basically comprises a dielectric block formed in a prism state, a clad layer that is formed on a face of the dielectric block, a light wave guide layer that is formed on the clad layer and comes into contact with a sample solution, a light source for generating a light beam, an optical system for allowing the above light beam to enter the dielectric block at various angles so that total reflection conditions can be obtained at the interface between the dielectric block and the clad layer, and a light-detecting means for detecting the excitation state of waveguide mode, that is, the state of attenuated total reflection, by measuring the intensity of the light beam totally reflected at the above interface.

In the leaking mode measurement device with the above structure, if a light beam is caused to enter the clad layer via the dielectric block at an incident angle greater than or equal to a total reflection angle, only light having a specific wave number that has entered at a specific incident angle is transmitted in a waveguide mode into the light wave guide layer, after the light beam has penetrated the clad layer. Thus, when the waveguide mode is excited, almost all forms of incident light are taken into the light wave guide layer, and thereby the state of attenuated total reflection occurs, in which the intensity of the totally reflected light is sharply decreased at the above interface. Since the wave number of a waveguide light depends on the refractive index of a measured substance placed on the light wave guide layer, the refractive index of the measurement substance or the properties of the measured substance associated therewith can be analyzed by determining the above specific incident angle causing the attenuated total reflection.

In this leaking mode measurement device also, the above-described array-form light-detecting means can be used to detect the position of a dark line generated in a reflected light due to attenuated total reflection. In addition, the above-described differentiating means can also be applied in combination with the above means.

The above-described surface plasmon measurement device or leaking mode measurement device may be used in random screening to discover a specific substance binding to a desired sensing substance in the field of research for development of new drugs or the like. In this case, a sensing substance is immobilized as the above-described measured substance on the above thin film layer (which is a metal film in the case of a surface plasmon measurement device, and is a clad layer and a light guide wave layer in the case of a leaking mode measurement device), and a sample solution obtained by dissolving various types of test substance in a solvent is added to the sensing substance. Thereafter, the above-described attenuated total reflection angle (θSP) is measured periodically when a certain period of time has elapsed.

If the test substance contained in the sample solution is bound to the sensing substance, the refractive index of the sensing substance is changed by this binding over time. Accordingly, the above attenuated total reflection angle (θSP) is measured periodically after the elapse of a certain time, and it is determined whether or not a change has occurred in the above attenuated total reflection angle (θSP), so that a binding state between the test substance and the sensing substance is measured. Based on the results, it can be determined whether or not the test substance is a specific substance binding to the sensing substance. Examples of such a combination between a specific substance and a sensing substance may include an antigen and an antibody, and an antibody and an antibody. More specifically, a rabbit anti-human IgG antibody is immobilized as a sensing substance on the surface of a thin film layer, and a human IgG antibody is used as a specific substance.

It is to be noted that in order to measure a binding state between a test substance and a sensing substance, it is not always necessary to detect the angle itself of an attenuated total reflection angle (θSP). For example, a sample solution may be added to a sensing substance, and the amount of an attenuated total reflection angle (θSP) changed thereby may be measured, so that the binding state can be measured based on the magnitude by which the angle has changed. When the above-described array-form light-detecting means and differentiating means are applied to a measurement device using attenuated total reflection, the amount by which a derivative value has changed reflects the amount by which the attenuated total reflection angle (θSP) has changed. Accordingly, based on the amount by which the derivative value has changed, a binding state between a sensing substance and a test substance can be measured (Japanese Patent Application No. 2000-398309 filed by the present applicant). In a measuring method and a measurement device using such attenuated total reflection, a sample solution consisting of a solvent and a test substance is added dropwise to a cup- or petri dish-shaped measurement chip wherein a sensing substance is immobilized on a thin film layer previously formed at the bottom, and then, the above-described amount by which an attenuated total reflection angle (θSP) has changed is measured.

Moreover, Japanese Patent Laid-Open No. 2001-330560 describes a measurement device using attenuated total reflection, which involves successively measuring multiple measurement chips mounted on a turntable or the like, so as to measure many samples in a short time.

When the biosensor of the present invention is used in surface plasmon resonance analysis, it can be applied as a part of various surface plasmon measurement devices described above.

The present invention will be further specifically described in the following examples. However, the examples are not intended to limit the scope of the present invention.

### EXAMPLES

### Example A-1

In the present example, various types of activators were allowed to act on a carboxyl group existing on the surface of a hydrophobic polymer applied onto a gold surface, and the presence or absence of generation of bubbles and the lifetime of an active ester were examined.

(1) Production of Substrate with Gold Surface

Films were formed on a glass substrate with a size of 8 mm long×80 mm wide×0.5 mm thick, using a parallel plate spputering device used for 6-inch objects (SH-550; manufactured by Ulvac, Inc.), such that chromium with a thickness of 1 nm was placed on the substrate and that gold with a thickness of 50 nm was further placed on the chromium. This substrate was then treated with a Model-208 UV-ozone cleaning system (TECHNOVISION INC.) for 30 minutes, so as to produce a substrate with a gold surface.

(2) Preparation of Coating Solution

1.5 g of poly(3,3,4,4,5,5,6,6,7,7,8,8,8-tridecafluorooctyl acrylate)-poly(benzyl methacrylate) copolymer (monomer weight ratio: 4/6; weight-average molecular weight: 30,000; hereinafter abbreviated as polymer A) was dissolved in methyl isobutyl ketone. Thereafter, methyl isobutyl ketone was added thereto to a liquid amount of 100 ml. This polymer A solution was filtrated with a 0.45-μm filter, so as to prepare coating solution A. Methyl isobutyl ketone used herein had previously been subjected to a dehydration treatment for 16 hours using molecular sieves 4A 1/16.

(3) Production of Polymer A-Coated Chip

The aforementioned substrate was placed in an aluminum container with a size of 40 mm long×120 mm wide×20 mm thick, having a hermetically closed structure. This aluminum container was fixed on the inner cup of a spin-coater equipped with a hermetically closed inner cup (MODEL SC408; manufactured by Nanotech), such that the glass substrate was located at a position of 135 mm from the center and the tangential direction of a circular arc became a long axis. 100 μl of coating solution A was added dropwise onto the glass substrate, using a micropipette, so that the entire surface of the glass substrate was coated with coating solution A. The aluminum container was hermetically closed, and it was then rotated at 200 rpm. 60 seconds later, the rotation was terminated. The substrate was left at rest in the hermetically closed container for 5 minutes, and it was then removed from the container. Thereafter, the substrate was dried at room temperature under ordinary pressure overnight, so as to obtain a polymer A-coated chip.

(4) Production of Biosensor Chip

The thus produced polymer A-coated chip was immersed for 2 hours in a 1 N NaOH aqueous solution that was kept at 60° C., and it was then washed with running pure water, so as to produce a biosensor chip, into the polymer A-coated layer surface of which a COOH group was introduced. Film thickness distribution in the center of the substrate was measured by ellipsometry (In-Situ Ellipsometer MAUS-101; manufactured by Five Lab Co., Ltd.) at a width of 75 mm at intervals of 0.1 mm in the horizontal direction. As a result, the mean film thickness was found to be 20 nm. The surface of the polymer A layer of the sensor chip was observed using a scanning electron microscope (S-5200; Hitachi Technologies) at an acceleration voltage of 0.5 kV. As a result, no defects were detected in the polymer A layer.

(5) Evaluation of Performance of Activator (the Presence or Absence of Generation of Bubbles and the Lifetime of Active Ester)

In a laboratory that was kept at 25° C., the produced sensor chip was placed in a test tube, and extra pure water (MilliQ) was then added thereto, such that the sensor chip as a whole can be immersed therein. Each of carboxylic acid activators (compounds A1 to A15 and an EDC/NHS mixture) was added thereto to a concentration of 1 mM, and generation of bubbles was visually observed.

Moreover, the sensor chip was immersed in an aqueous solution containing each of carboxylic acid activators A1 to A15 and the EDC/NHS mixture at a concentration of 1 mM, for 1 hour, 4 hours, or 16 hours in a condition of 25° C. Thereafter, the sensor chip was washed with extra pure water 5 times, and it was then immersed in an aqueous solution containing Cy-5-hydrazide (manufactured by Amersham) used as a fluorescent dye (0.50 mg/L) for 30 minutes. Thereafter, the sensor chip was washed with extra pure water 5 times, so as to eliminate unreacted Cy-5-hydrazide from the surface of the sensor chip. Thereafter, the relative value of fluorescence intensity on the surface of the sensor chip was compared using a fluoroimage analyzer (FLA8000; manufactured by Fuji Photo Film Co., Ltd.) (excitation wavelength: 635 nm; measurement wavelength: 675 nm). Fluorescence was observed on the surface of the sensor chip, only when an active ester on the sensor chip surface was reacted with Cy-5-hydrazide. Thus, the present means may constitute a useful means for evaluating the lifetime of an active ester. The obtained results are shown in Table 1.

TABLE 1

| | | | | Fluorescence intensity | | | |
|---|---|---|---|---|---|---|---|
| Sample | | Manufacturer | Generation | (relative value) | | | |
| No. | Activator | (Code) | of bubbles | 1 hr | 4 hrs | 16 hrs | Remarks |
| 1 | Compound A1 (X = PF₆) | Kokusan Chemical | None | ◯ | ◯ | Δ | The present |
| | | (HBTU) | | | | | invention |
| 2 | Compound A2 (X = PF₆) | Aldrich (HBPyU) | None | ◯ | ◯ | Δ | The present |
| | | | | | | | invention |
| 3 | Compound A3 (X = PF₆) | Aldrich (HBPipU) | None | ◯ | ◯ | Δ | The present |
| | | | | | | | invention |
| 4 | Compound A4 (X = PF₆) | Synthesized in | None | ◯ | ◯ | Δ | The present |
| | | accordance with | | | | | invention |
| | | Publication 1 | | | | | |
| | | (BOI) | | | | | |
| 5 | Compound A5 (X = BF₄) | Aldrich (TDBTU) | None | ◯ | ◯ | Δ | The present |
| | | | | | | | invention |
| 6 | Compound A6 (X = PF₆) | Aldrich (HATU) | None | ◯ | ◯ | Δ | The present |
| | | | | | | | invention |
| 7 | Compound A7 (X = PF₆) | Sakai Kogyo | None | ◯ | ◯ | Δ | The present |
| | | (HCTU) | | | | | invention |
| 8 | Compound A8 (X = BF₄) | Sakai Kogyo | None | ◯ | ◯ | ◯ | The present |
| | | (TNTU) | | | | | invention |
| 9 | Compound A9 (X = BF₄) | Sakai Kogyo | None | ◯ | ◯ | Δ | The present |
| | | (TPTU) | | | | | invention |
| 10 | Compound A10 | Sakai Kogyo | None | ◯ | ◯ | Δ | The present |
| | (X = PF₆) | (PFTU) | | | | | invention |
| 11 | Compound A11 | Kokusan Chemical | None | ◯ | ◯ | Δ | The present |
| | (X = PF₆) | (BOP) | | | | | invention |
| 12 | Compound A12 | Synthesized in | None | ◯ | ◯ | Δ | The present |
| | (X = PF₆) | accordance with | | | | | invention |
| | | Publication 2 | | | | | |
| | | (AOP) | | | | | |
| 13 | Compound A13 | Kokusan Chemical | None | ◯ | ◯ | ◯ | The present |
| | (X = PF₆) | (PyBOP) | | | | | invention |
| 14 | Compound A14 | Aldrich (PyAOP) | None | ◯ | Δ | Δ | The present |
| | (X = PF₆) | | | | | | invention |
| 15 | Compound A15 (X = Cl) | Kokusan Chemical | None | ◯ | Δ | Δ | The present |
| | | (DMT-MM) | | | | | invention |
| 16 | EDC/NHS | Dojindo | Yes | ◯ | Δ | X | Comparative |
| | | Laboratories | | | | | example |

Publication 1: L. A. Carpino et al., J. Chem. Soc. Chem. Commun., 1994, 201.
Publication 2: Y. Kiso et al., Chem. Pharm. Bull., 38, 270 (1990).

Generation of bubbles was observed as a result of the reaction in the EDC/NHS mixture system (Sample No. 16) used as a comparative example. In addition, 16 hours after the reaction, almost all the active esters disappeared from sample No. 16. In contrast, in the present invention using compounds A1 to A15 (sample Nos. 1 to 15), generation of bubbles was not observed, and even 16 hours after the reaction, active esters remained. In particular, it was shown that active esters obtained from compound A8 and compound A15 were stable.

### Example A-2

In the present example, various types of activators were allowed to act on a carboxyl group existing on the surface of a hydrophobic polymer applied onto a gold surface, and the performance as a biosensor chip was examined.

(1) Non-Specific Adsorption Prevention Performance

A sensor chip was produced in accordance with the operations described in (1) to (4) of Example A-1. An aqueous solution containing a 0.1 mM carboxylic acid activator (each of compounds A1 to A15 and an EDC/NHS mixture) was allowed to come into contact with the produced sensor chip for 30 minutes. Thereafter, the sensor chip was washed with a 50 mM acetate buffer (pH 4.5; manufactured by Biacore). Subsequently, an ethanolamine-HCl solution (1 M, pH 8.5) was allowed to come into contact with the sensor chip for 30 minutes, and it was then washed with a 50 mM acetate buffer (pH 4.5), so as to block a COOH group onto the surface. Each sensor chip produced by the aforementioned procedures was placed in a surface plasmon resonance measurement device (the SPR resonance device shown in FIG. 5 of Applied Spectroscopy, 42(8), 1375-1379 (1988)). When the central position to which a laser beam was applied was in the vertical direction, the sensor chip was set in the center. When the above position was in the horizontal direction, the sensor chip was set at 40 mm from the edge. A structural member made from polypropylene was placed on the chip, so as to form a cell with a size of 1 mm long (vertical direction), 7.5 mm wide (horizontal direction), and 1 mm thick. The inside of this measurement cell was filled with an HBS-EP buffer, and measurement was then initiated. The HBS-EP buffer consists of 0.01 mol/l HEPES (N-2-hydroxyethylpiperazine-N′-2-ethanesulfonic acid) (pH 7.4), 0.15 mol/l NaCl, 0.003 mol/l EDTA, and 0.005% by weight of Surfactant P20. The inside of the cell was replaced with a BSA solution (2 mg/ml, HBS-P buffer (manufactured by Biacore; pH 7.4)) or an avidin solution (2 mg/ml, HBS-P buffer), and it was then left at rest for 10 minutes. Thereafter, it was washed with an HBS-EP buffer. The amount of resonance signals (RU value) changed 3 minutes after the washing was measured. The amount by which resonance signals (RU value) obtained before the addition of each protein and 3 minutes after washing it with the buffer had changed was defined as the amount of non-specific adsorption of each protein. It was evaluated with a relative value with respect to the amount changed, obtained from an unmodified gold surface substrate (sample No. 33).

(2) Interaction Between Protein and Test Compound

Neutral avidin (manufactured by PIERCE) was immobilized on a sensor chip, and the interaction of neutral avidin with D-biotin (manufactured by Nacalai Tesque) was measured by the following method.

A sensor chip was produced in accordance with the operations described in (1) to (4) of Example A-1. An aqueous solution containing a 0.1 mM carboxylic acid activator (each of compounds A1 to A15 and an EDC/NHS mixture) was allowed to come into contact with the produced sensor chip for 30 minutes. Thereafter, the sensor chip was washed with an HBS-N buffer (pH 7.4; manufactured by Biacore). The HBS-N buffer consists of 0.01 mol/l HEPES (N-2-hydroxyethylpiperazine-N′-2-ethanesulfonic acid) (pH 7.4) and 0.15 mol/l NaCl. Subsequently, the sensor chip was placed in the surface plasmon resonance measurement device of the present invention. When the central position to which a laser beam was applied was in the vertical direction, the sensor chip was set in the center. When the above position was in the horizontal direction, the sensor chip was set at 40 mm from the edge. A structural member made from polypropylene was placed on the chip, so as to form a cell with a size of 1 mm long (vertical direction), 7.5 mm wide (horizontal direction), and 1 mm thick. The inside of this cell was substituted with a neutral avidin solution (100 μg/ml, an HBS-N buffer), and it was then left at rest for 30 minutes. Thereafter, the above solution was substituted with an HBS-N buffer. By the aforementioned operations, N-avidin was immobilized on the surface of the sensor chip via a covalent bond.

With regard to a sensor chip (sample No. 32) activated with EDC/NHS, the amount by which resonance signals (RU value) obtained before the addition of neutral avidin and 3 minutes after completion of substitution of the buffer had changed was defined as a reference binding amount. The amount by which resonance signals (RU value) obtained before the addition of neutral avidin and 3 minutes after completion of substitution with an HBS-N buffer had changed (namely, the binding amount of neutral avidin) of each of sensor chips (samples Nos. 17 to 31) activated with compounds A1 to A15 was evaluated in the form of a relative value with respect to the above reference value.

Thereafter, the inside of the cell was substituted with an ethanolamine-HCl solution (1 M, pH 8.5), and then substituted with an HBS-N buffer, so as to block remaining COOH groups that had not been reacted with neutral avidin. Subsequently, the inside of the cell was substituted with D-biotin (1 μg/ml, an HBS-N buffer), and it was then left at rest for 10 minutes. Thereafter, it was substituted with an HBS-N buffer.

With regard to a sensor chip (sample No. 32) activated with EDC/NHS, the amount by which resonance signals (RU value) obtained before the addition of D-biotin and 3 minutes after washing had changed was defined as a reference binding amount. The amount by which resonance signals (RU value) obtained before the addition of D-biotin and 3 minutes after washing had changed (namely, the binding amount of D-biotin) of each of sensor chips (samples Nos. 17 to 31) activated with compounds A1 to A15 was evaluated in the form of a relative value with respect to the above reference value. The obtained results are shown in Table 2.

TABLE 2

| | | Amount of | | | | |
|---|---|---|---|---|---|---|
| | | non-specific | | | | |
| Sample | | adsorption | | Binding amount | | |
| No. | Activator | BSA | Avidin | N-Avidin | D-Biotin | Remarks |
| 17 | Compound A1 | 0.1 | 0.1 | 5.7 | 6.3 | The present invention |
| | (X = PF₆) | | | | | |
| 18 | Compound A2 | 0.1 | 0.1 | 6.3 | 5.9 | The present invention |
| | (X = PF₆) | | | | | |
| 19 | Compound A3 | 0.1 | 0.1 | 7.2 | 6.6 | The present invention |
| | (X = PF₆) | | | | | |
| 20 | Compound A4 | 0.1 | 0.1 | 6.4 | 6.6 | The present invention |
| | (X = PF₆) | | | | | |
| 21 | Compound A5 | 0.1 | 0.1 | 6.8 | 7.1 | The present invention |
| | (X = BF₄) | | | | | |
| 22 | Compound A6 | 0.1 | 0.1 | 5.9 | 6.6 | The present invention |
| | (X = PF₆) | | | | | |
| 23 | Compound A7 | 0.1 | 0.1 | 6.8 | 5.7 | The present invention |
| | (X = PF₆) | | | | | |
| 24 | Compound A8 | 0.1 | 0.1 | 10.5 | 9.8 | The present invention |
| | (X = BF₄) | | | | | |
| 25 | Compound A9 | 0.1 | 0.1 | 4.3 | 5.0 | The present invention |
| | (X = BF₄) | | | | | |
| 26 | Compound A10 | 0.1 | 0.1 | 3.9 | 4.4 | The present invention |
| | (X = PF₆) | | | | | |
| 27 | Compound A11 | 0.1 | 0.1 | 6.9 | 7.5 | The present invention |
| | (X = PF₆) | | | | | |
| 28 | Compound A12 | 0.1 | 0.1 | 7.2 | 6.6 | The present invention |
| | (X = PF₆) | | | | | |
| 29 | Compound A13 | 0.1 | 0.1 | 7.9 | 7.2 | The present invention |
| | (X = PF₆) | | | | | |
| 30 | Compound A14 | 0.1 | 0.1 | 7.2 | 6.6 | The present invention |
| | (X = PF₆) | | | | | |
| 31 | Compound A15 | 0.1 | 0.1 | 13.2 | 14.2 | The present invention |
| | (X = Cl) | | | | | |
| 32 | EDC/NHS | 0.5 | 0.6 | 1.0 | 1.0 | Comparative example |
| 33 | Unmodified gold | 1.0 | 1.0 | — | — | Comparative example |
| | surface | | | | | |

When the sensor chip (sample No. 32) activated with EDCNHS used as a comparative example was allowed to react with ethanolamine, it had ability to prevent non-specific adsorption, but the degree of the ability was not sufficient when compared with that of the unmodified gold surface (sample No. 33). In contrast, when the sensor chips (samples Nos. 17 to 31) activated with compounds A1 to A15 in the present invention were allowed to react with ethanolamine, it had sufficient ability to prevent non-specific adsorption.

On the other hand, in the experiment in which carboxylic acid on the sensor chip surface is activated and neutral avidin is allowed to bind thereto via an amide bond, in the case where the sensor chips (samples Nos. 17 to 31) activated with compounds A1 to A15 in the present invention was allowed to react with neutral avidin, a larger amount of neutral avidin can be immobilized on the surface thereof via amide bond than the case where the sensor chip (sample No. 32) activated with EDC/NHS used as a comparative example was allowed to react with neutral avidin. As a result, it was shown that a larger amount of D-biotin could be detected. Such effect could significantly be obtained, particularly when compound A8 and compound A15 were used.

### Example B-1

In the present example, various types of activators were allowed to act on a carboxyl group existing on the surface of a hydrophobic polymer applied onto a gold surface, and the lifetime of an active ester was examined.

(1) Production of Substrate with Gold Surface

Films were formed on a glass substrate with a size of 8 mm long×80 mm wide×0.5 mm thick, using a parallel plate spputering device used for 6-inch objects (SH-550; manufactured by Ulvac, Inc.), such that chromium with a thickness of 1 nm was placed on the substrate and that gold with a thickness of 50 nm was further placed on the chromium. This substrate was then treated with a Model-208 UV-ozone cleaning system (TECHNOVISION INC.) for 30 minutes, so as to produce a substrate with a gold surface.

(2) Preparation of Coating Solution

1.5 g of poly(3,3,4,4,5,5,6,6,7,7,8,8,8-tridecafluorooctyl acrylate)-poly(benzyl methacrylate) copolymer (monomer weight ratio: 4/6; weight-average molecular weight: 30,000; hereinafter abbreviated as polymer A) was dissolved in methyl isobutyl ketone. Thereafter, methyl isobutyl ketone was added thereto to a liquid amount of 100 ml. This polymer A solution was filtrated with a 0.45-μm filter, so as to prepare coating solution A. Methyl isobutyl ketone used herein had previously been subjected to a dehydration treatment for 16 hours using molecular sieves 4A 1/16.

(3) Production of Polymer A-Coated Chip

The aforementioned substrate was placed in an aluminum container with a size of 40 mm long×120 mm wide×20 mm thick, having a hermetically closed structure. This aluminum container was fixed on the inner cup of a spin-coater equipped with a hermetically closed inner cup (MODEL SC408; manufactured by Nanotech), such that the glass substrate was located at a position of 135 mm from the center and the tangential direction of a circular arc became a long axis. 100 μl of coating solution A was added dropwise onto the glass substrate, using a micropipette, so that the entire surface of the glass substrate was coated with coating solution A. The aluminum container was hermetically closed, and it was then rotated at 200 rpm. 60 seconds later, the rotation was terminated. The substrate was left at rest in the hermetically closed container for 5 minutes, and it was then removed from the container. Thereafter, the substrate was dried at room temperature under ordinary pressure overnight, so as to obtain a polymer A-coated chip.

(4) Production of Biosensor Chip

The thus produced polymer A-coated chip was immersed for 2 hours in a 1 N NaOH aqueous solution that was kept at 60° C., and it was then washed with running pure water, so as to produce a biosensor chip, into the polymer A-coated layer surface of which a COOH group was introduced. Film thickness distribution in the center of the substrate was measured by ellipsometry (In-Situ Ellipsometer MAUS-101; manufactured by Five Lab Co., Ltd.) at a width of 75 mm at intervals of 0.1 mm in the horizontal direction. As a result, the mean film thickness was found to be 20 nm. The surface of the polymer A layer of the sensor chip was observed using a scanning electron microscope (S-5200; Hitachi Technologies) at an acceleration voltage of 0.5 kV. As a result, no defects were detected in the polymer A layer.

(5) Evaluation of Performance of Activator (Life Time of Active Ester)

In a laboratory that was kept at 25° C., hydrochloride as compound B1 (Dojindo Laboratory) and an ester-forming compound (any one of compounds B4 to B19 and NHS) were dissolved in extra pure water (MilliQ) to each concentration of 1 mM. The sensor chip produced in (4) above was immersed in the obtained solution. 1 hour, 4 hours, and 16 hours later, the sensor chip was washed with extra pure water 5 times at 25° C., and it was then immersed in an aqueous solution containing Cy-5-hydrazide (manufactured by Amersham) used as fluorescent dye (0.50 mg/L) for 30 minutes. Thereafter, the sensor chip was washed with extra pure water 5 times, so as to eliminate unreacted Cy-5-hydrazide from the surface of the sensor chip. Thereafter, the relative value of fluorescence intensity on the surface of the sensor chip was compared using a fluoroimage analyzer (FLA8000; manufactured by Fuji Photo Film Co., Ltd.) (excitation wavelength: 635 nm; measurement wavelength: 675 nm). Fluorescence was observed on the surface of the sensor chip, only when an active ester on the sensor chip surface was reacted with Cy-5-hydrazide. Thus, the present means may constitute a useful means for evaluating the life time of an active ester. The obtained results are shown in Table 3.

TABLE 3

| | | Fluorescence intensity | | | |
|---|---|---|---|---|---|
| Sample | | (relative value) | | | |
| No. | Activator | 1 hr | 4 hrs | 16 hrs | Remarks |
| 1 | Compound B4 | ◯ | ◯ | ◯ | The present |
| | | | | | invention |
| 2 | Compound B5 | ◯ | ◯ | ◯ | The present |
| | | | | | invention |
| 3 | Compound B6 | ◯ | ◯ | ◯ | The present |
| | | | | | invention |
| 4 | Compound B7 | ◯ | ◯ | ◯ | The present |
| | | | | | invention |
| 5 | Compound B8 | ◯ | ◯ | ◯ | The present |
| | | | | | invention |
| 6 | Compound B9 | ◯ | ◯ | Δ | The present |
| | | | | | invention |
| 7 | Compound B10 | ◯ | ◯ | Δ | The present |
| | | | | | invention |
| 8 | Compound B11 | ◯ | ◯ | ◯ | The present |
| | | | | | invention |
| 9 | Compound B12 | ◯ | ◯ | ◯ | The present |
| | | | | | invention |
| 10 | Compound B13 | ◯ | ◯ | Δ | The present |
| | | | | | invention |
| 11 | Compound B14 | ◯ | ◯ | Δ | The present |
| | | | | | invention |
| 12 | Compound B15 | ◯ | ◯ | Δ | The present |
| | | | | | invention |
| 13 | Compound B16 | ◯ | ◯ | ◯ | The present |
| | | | | | invention |
| 14 | Compound B17 | ◯ | ◯ | Δ | The present |
| | | | | | invention |
| 15 | Compound B18 | ◯ | ◯ | ◯ | The present |
| | | | | | invention |
| 16 | Compound B19 | ◯ | ◯ | ◯ | The present |
| | | | | | invention |
| 17 | NHS | ◯ | Δ | X | Comparative |
| | | | | | example |

An NHS ester (sample No. 17) used as a comparative example completely lost its activity 16 hours after the reaction. In contrast, compounds B4 to B19 (sample Nos. 1 to 16) in the present invention maintained their activity even 16 hours after the reaction.

### Example B-2

In the present example, various types of activators were allowed to act on a carboxyl group existing on the surface of a hydrophobic polymer applied onto a gold surface, and the performance as a biosensor chip was examined.

(1) Non-Specific Adsorption Prevention Performance

A sensor chip was produced in accordance with the operations described in (1) to (4) of Example B-1. An aqueous solution containing 1 mM compound B1 (Dojindo Laboratory) and 1 mM an ester-forming compound (any one of compounds B4 to B19 and NHS) was allowed to come into contact with the produced sensor chip for 30 minutes. Thereafter, the sensor chip was washed with a 50 mM acetate buffer (pH 4.5; manufactured by Biacore). Subsequently, an ethanolamine-HCl solution (1 M, pH 8.5) was allowed to come into contact with the sensor chip for 30 minutes, and it was then washed with a 50 mM acetate buffer (pH 4.5), so as to block a COOH group onto the surface. Each sensor chip produced by the aforementioned procedures was placed in a surface plasmon resonance measurement device (the SPR resonance device shown in FIG. 5 of Applied Spectroscopy, 42(8), 1375-1379 (1988)). When the central position to which a laser beam was applied was in the vertical direction, the sensor chip was set in the center. When the above position was in the horizontal direction, the sensor chip was set at 40 mm from the edge. A structural member made from polypropylene was placed on the chip, so as to form a cell with a size of 1 mm long (vertical direction), 7.5 mm wide (horizontal direction), and 1 mm thick. The inside of this measurement cell was filled with an HBS-EP buffer, and measurement was then initiated. The HBS-EP buffer consists of 0.01 mol/l HEPES (N-2-hydroxyethylpiperazine-N′-2-ethanesulfonic acid) (pH 7.4), 0.15 mol/l NaCl, 0.003 mol/l EDTA, and 0.005% by weight of Surfactant P20. The inside of the cell was replaced with a BSA solution (2 mg/ml, HBS-P buffer (manufactured by Biacore; pH 7.4)) or an avidin solution (2 mg/ml, HBS-P buffer), and it was then left at rest for 10 minutes. Thereafter, it was washed with an HBS-EP buffer. The amount of resonance signals (RU value) changed 3 minutes after the washing was measured. The amount by which resonance signals (RU value) obtained before the addition of each protein and 3 minutes after washing it with the buffer had changed was defined as the amount of non-specific adsorption of each protein. It was evaluated with a relative value with respect to the amount changed, obtained from an unmodified gold surface substrate (sample No. 35).

(2) Interaction Between Protein and Test Compound

Neutral avidin (manufactured by PIERCE) was immobilized on a sensor chip, and the interaction of neutral avidin with D-biotin (manufactured by Nacalai Tesque) was measured by the following method.

A sensor chip was produced in accordance with the operations described in (1) to (4) of Example B-1. An aqueous solution containing 0.1 mM compound B1 and 0.1 mM an ester-forming compound (any one of compounds B4 to B19 and NHS) was allowed to come into contact with the produced sensor chip for 30 minutes. Thereafter, the sensor chip was washed with an HBS-N buffer (pH 7.4; manufactured by Biacore). The HBS-N buffer consists of 0.01 mol/l HEPES (N-2-hydroxyethylpiperazine-N′-2-ethanesulfonic acid) (pH 7.4) and 0.15 mol/l NaCl. Subsequently, the sensor chip was placed in the surface plasmon resonance measurement device of the present invention. When the central position to which a laser beam was applied was in the vertical direction, the sensor chip was set in the center. When the above position was in the horizontal direction, the sensor chip was set at 40 mm from the edge. A structural member made from polypropylene was placed on the chip, so as to form a cell with a size of 1 mm long (vertical direction), 7.5 mm wide (horizontal direction), and 1 mm thick. The inside of this cell was substituted with a neutral avidin solution (100 μg/ml, an HBS-N buffer), and it was then left at rest for 30 minutes. Thereafter, the above solution was substituted with an HBS-N buffer. By the aforementioned operations, N-avidin was immobilized on the surface of the sensor chip via a covalent bond.

With regard to a sensor chip (sample No. 34) esterified with NHS, the amount by which resonance signals (RU value) obtained before the addition of neutral avidin and 3 minutes after completion of substitution of the buffer had changed was defined as a reference binding amount. The amount by which resonance signals (RU value) obtained before the addition of neutral avidin and 3 minutes after completion of substitution with an HBS-N buffer had changed (namely, the binding amount of neutral avidin) of each of sensor chips (samples Nos. 18 to 33) esterified with compounds B4 to B19 was evaluated in the form of a relative value with respect to the above reference value.

Thereafter, the inside of the cell was substituted with an ethanolamine-HCl solution (1 M, pH 8.5), and then substituted with an HBS-N buffer, so as to block remaining COOH groups that had not been reacted with neutral avidin. Subsequently, the inside of the cell was substituted with D-biotin (1 μg/ml, an HBS-N buffer), and it was then left at rest for 10 minutes. Thereafter, it was substituted with an HBS-N buffer.

With regard to a sensor chip (sample No. 34) esterified with NHS, the amount by which resonance signals (RU value) obtained before the addition of D-biotin and 3 minutes after washing had changed was defined as a reference binding amount. The amount by which resonance signals (RU value) obtained before the addition of D-biotin and 3 minutes after washing had changed (namely, the binding amount of D-biotin) of each of sensor chips (samples Nos. 18 to 33) esterified with compounds B4 to B19 was evaluated in the form of a relative value with respect to the above reference value. The obtained results are shown in Table 4.

TABLE 4

| | | Amount of | | | | |
|---|---|---|---|---|---|---|
| | | non-specific | | | | |
| Sample | Ester-forming | adsorption | | Binding amount | | |
| No. | compound | BSA | Avidin | N-Avidin | D-Biotin | Remarks |
| 18 | Compound B4 | 0.1 | 0.1 | 13.8 | 11.3 | The present invention |
| 19 | Compound B5 | 0.1 | 0.1 | 16.2 | 12.8 | The present invention |
| 20 | Compound B6 | 0.1 | 0.1 | 9.2 | 10.7 | The present invention |
| 21 | Compound B7 | 0.1 | 0.1 | 12.3 | 11.4 | The present invention |
| 22 | Compound B8 | 0.1 | 0.1 | 9.9 | 10.3 | The present invention |
| 23 | Compound B9 | 0.1 | 0.1 | 3.6 | 4.8 | The present invention |
| 24 | Compound B10 | 0.1 | 0.1 | 5.7 | 6.3 | The present invention |
| 25 | Compound B11 | 0.1 | 0.1 | 12.3 | 10.6 | The present invention |
| 26 | Compound B12 | 0.1 | 0.1 | 8.2 | 9.1 | The present invention |
| 27 | Compound B13 | 0.1 | 0.1 | 5.8 | 4.8 | The present invention |
| 28 | Compound B14 | 0.1 | 0.1 | 4.3 | 4.6 | The present invention |
| 29 | Compound B15 | 0.1 | 0.1 | 3.6 | 4.1 | The present invention |
| 30 | Compound B16 | 0.1 | 0.1 | 9.3 | 8.6 | The present invention |
| 31 | Compound B17 | 0.1 | 0.1 | 3.9 | 4.6 | The present invention |
| 32 | Compound B18 | 0.1 | 0.1 | 8.9 | 9.2 | The present invention |
| 33 | Compound B19 | 0.1 | 0.1 | 9.7 | 9.1 | The present invention |
| 34 | NHS | 0.5 | 0.6 | 1.0 | 1.0 | Comparative example |
| 35 | Unmodified gold | 1.0 | 1.0 | — | — | Comparative example |
| | surface | | | | | |

When the sensor chip (sample No. 34) esterified with NHS used as a comparative example was allowed to react with ethanolamine, it had ability to prevent non-specific adsorption, but the degree of the ability was not sufficient when compared with that of the unmodified gold surface (sample No. 35). In contrast, when the sensor chips (samples Nos. 18 to 33) activated with compounds B4 to B19 in the present invention were allowed to react with ethanolamine, it had sufficient ability to prevent non-specific adsorption.

On the other hand, in the experiment in which carboxylic acid on the sensor chip surface is activated and neutral avidin is allowed to bind thereto via an amide bond, in the case where the sensor chips (samples Nos. 18 to 33) esterified with compounds B14 to B19 in the present invention was allowed to react with neutral avidin, a larger amount of neutral avidin can be immobilized on the surface thereof via amide bond than the case where the sensor chip (sample No. 34) activated with EDC/NHS used as a comparative example was allowed to react with neutral avidin. As a result, it was shown that a larger amount of D-biotin could be detected.

### EFECTS OF THE INVENTION

The present invention enables conversion of carboxylic acid into an active ester with no generation of air bubbles in the production of a biosensor, as well as stabilization of the obtained active ester. The use of the biosensor of the present invention enables suppression of the amount of non-specific adsorption on the sensor surface and improvement of the conventional binding amount of a physiologically active substance.

1. A biosensor, wherein a carboxyl group existing on the surface of a substrate thereof is activated with any one compound selected from the group consisting of an uronium salt represented by the following formula A1, a phosphonium salt represented by the following formula A2, and a triazine derivative represented by the following formula A3, so as to form a carboxylic acid amide group: 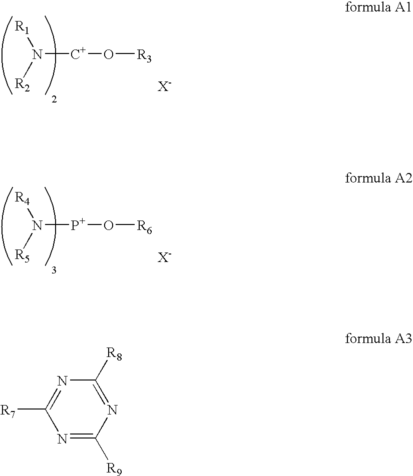
wherein, in the formula A1, each of R₁ and R₂ independently represents an alkyl group having 1 to 6 carbon atoms, or R₁ and R₂ together form an alkylene group having 2 to 6 carbon atoms, which forms a ring together with an N atom, R₃ represents an aromatic group having 6 to 20 carbon atoms or a heterocyclic group containing at least one heteroatom, and X⁻ represents an anion; in the formula A2, each of R₄ and R₅ independently represents an alkyl group having 1 to 6 carbon atoms, or R₄ and R₅ together form an alkylene group having 2 to 6 carbon atoms, which forms a ring together with an N atom, R₆ represents an aromatic group having 6 to 20 carbon atoms or a heterocyclic group containing at least one heteroatom, and X⁻ represents an anion; and in the formula A3, R₇ represents an onium group, and each of R₈ and R₉ independently represents an electron-donating group.

2. The biosensor of claim 1 wherein the uronium salt represented by the formula A1 is any one compound selected from the following compounds A1 to A10 wherein X⁻ represents an anion.

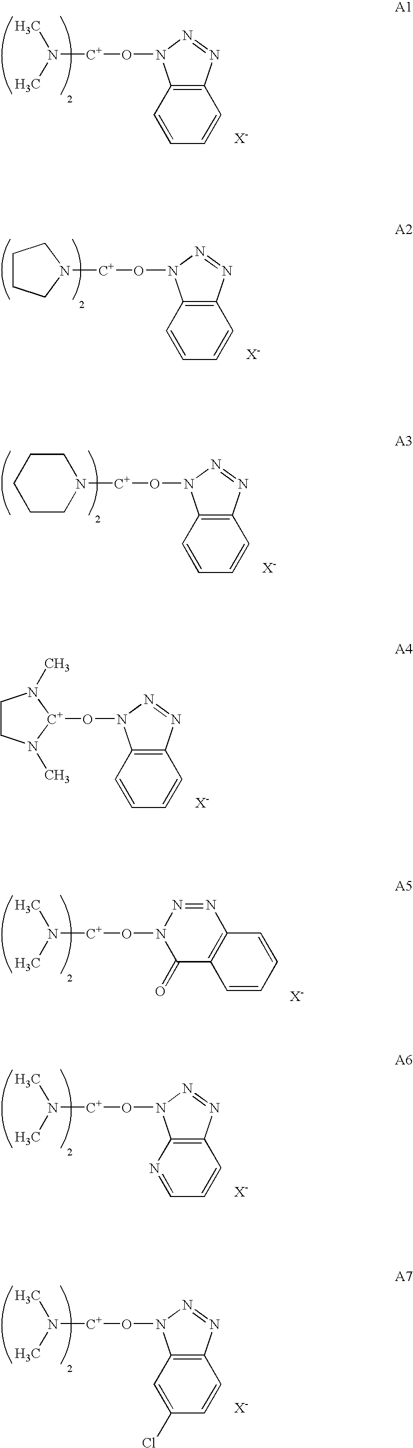

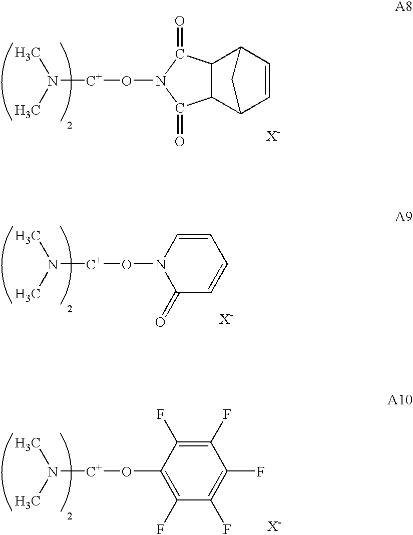

3. The biosensor of claim 1 wherein the phosphonium salt represented by the formula 2 is any one compound selected from the following compounds A11 to A14 wherein X⁻ represents an anion.

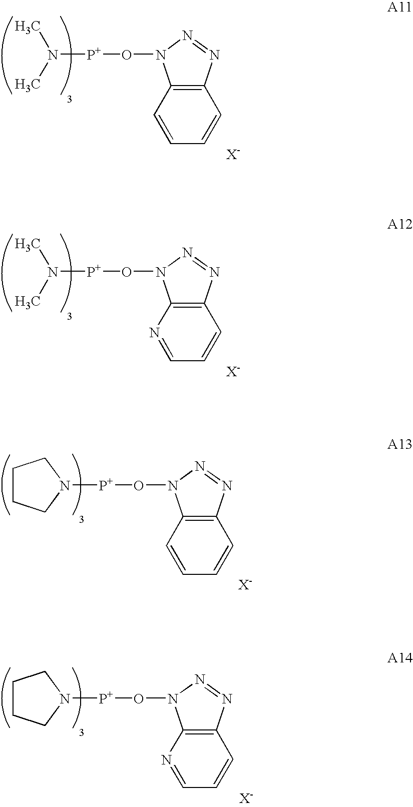

4. The biosensor of claim 1 wherein the triazine derivative represented by the formula A3 is the following compound A15 wherein X⁻ represents an anion.

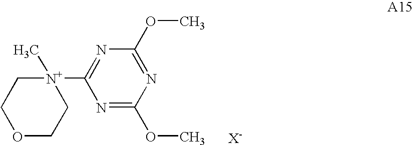

5. The biosensor of claim 1, which is used in surface plasmon resonance analysis.

6. A biosensor, wherein a carboxyl group existing on the surface of a substrate thereof is activated with a carbodiimide derivative or a salt thereof, and it is then converted into an ester using any one compound selected from the group consisting of a nitrogen-containing heteroaromatic compound having a hydroxyl group, a phenol derivative having an electron-withdrawing group, and an aromatic compound having a thiol group, followed by a reaction with, amine, so as to form a carboxylic acid amide group.

7. The biosensor of claim 6 wherein the carbodiimide derivative is any one of the following compounds B1 to B3.

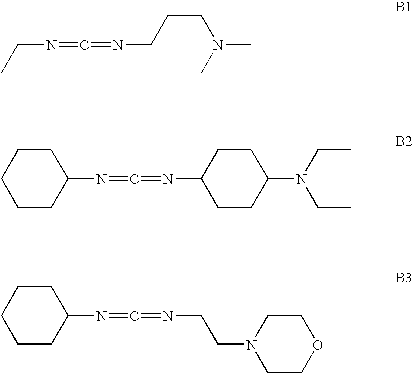

8. The biosensor of claim 6 wherein the nitrogen-containing heteroaromatic compound having a hydroxyl group is any one of the following compounds B4 to B12.

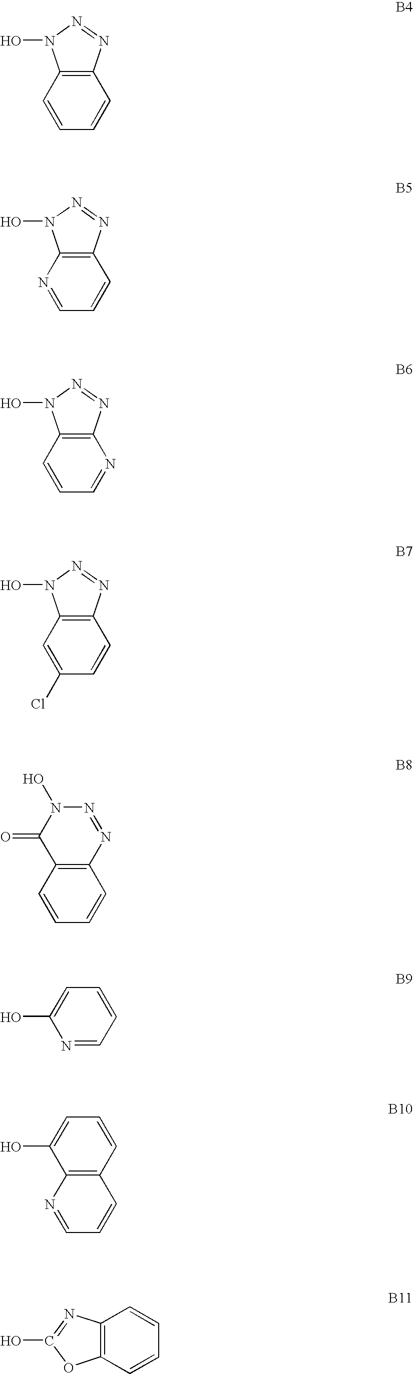

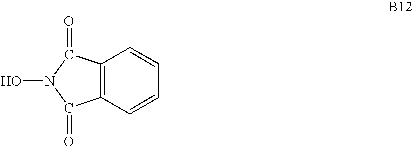

9. The biosensor of claim 6 wherein, in the phenol derivative having an electron-withdrawing group, the σ value of the electron-withdrawing group is 0.3 or greater.

10. The biosensor of claim 9 wherein the phenol derivative having an electron-withdrawing group is any one of the following compounds B13 to B16.

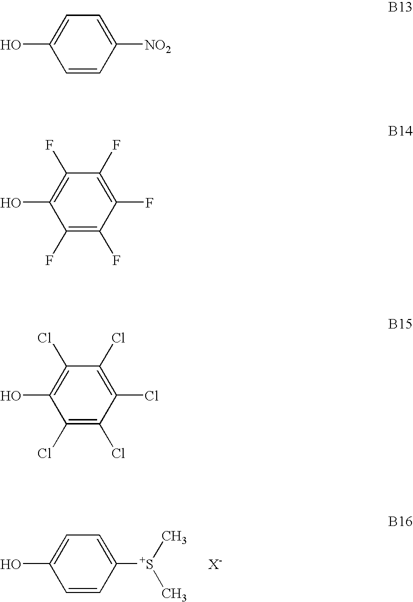

11. The biosensor of claim 6 wherein the aromatic compound having a thiol group is any one of the following compounds B17 to B19.

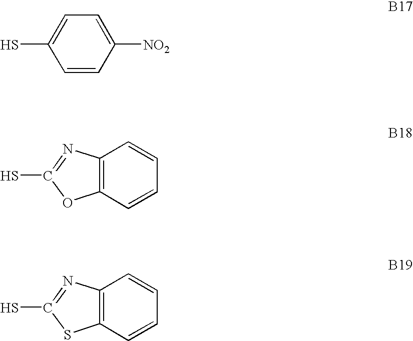

12. The biosensor of claim 6, which is used in surface plasmon resonance analysis.

13. A method for producing the biosensor of claim 1, which comprises a step of activating a substrate having a carboxyl group on its surface with any one compound selected from the group consisting of an uronium salt represented by the following formula A1, a phosphonium salt represented by the following formula A2, and a triazine derivative represented by the following formula A3, so as to form a carboxylic acid amide group: 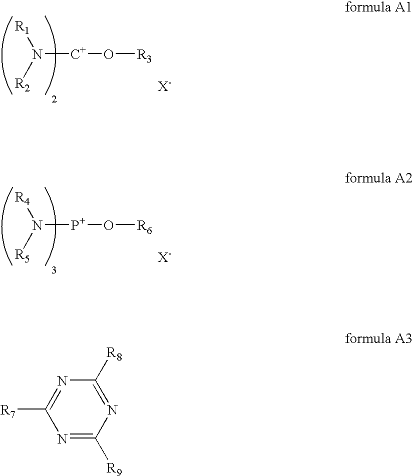
wherein, in the formula A1, each of R₁ and R₂ independently represents an alkyl group having 1 to 6 carbon atoms, or R₁ and R₂ together form an alkylene group having 2 to 6 carbon atoms, which forms a ring together with an N atom, R₃ represents an aromatic group having 6 to 20 carbon atoms or a heterocyclic group containing at least one heteroatom, and X⁻ represents an anion; in the formula A2, each of R₄ and R₅ independently represents an alkyl group having 1 to 6 carbon atoms, or R₄ and R₅ together form an alkylene group having 2 to 6 carbon atoms, which forms a ring together with an N atom, R₆ represents an aromatic group having 6 to 20 carbon atoms or a heterocyclic group containing at least one heteroatom, and X⁻ represents an anion; and in the formula A3, R₇ represents an onium group, and each of R₈ and R₉ independently represents an electron-donating group.

14. A method for producing the biosensor of claim 6, which comprises steps of activating a substrate having a carboxyl group on its surface with a carbodiimide derivative or a salt thereof, and then converting it into an ester using any one compound selected from the group consisting of a nitrogen-containing heteroaromatic compound having a hydroxyl group, a phenol derivative having an electron-withdrawing group, and an aromatic compound having a thiol group, followed by a reaction with amine, so as to form a carboxylic acid amide group.

15. A method for immobilizing a physiologically active substance on a biosensor, which comprises a step of allowing a physiologically active substance to come into contact with the biosensor of claim 1, so as to allow said physiologically active substance to bind to the surface of said biosensor via a covalent bond.

16. A method for immobilizing a physiologically active substance on a biosensor, which comprises a step of allowing a physiologically active substance to come into contact with the biosensor of claim 6, so as to allow said physiologically active substance to bind to the surface of said biosensor via a covalent bond.

17. A method for detecting or measuring a substance interacting with a physiologically active substance, which comprises a step of allowing a test substance to come into contact with the biosensor of claim 1 to the surface of which the physiologically active substance binds via a covalent bond.

18. The method of claim 17, wherein the substance interacting with the physiologically active substance is detected or measured by surface plasmon resonance analysis.

19. A method for detecting or measuring a substance interacting with a physiologically active substance, which comprises a step of allowing a test substance to come into contact with the biosensor of claim 6 to the surface of which the physiologically active substance binds via a covalent bond.

20. The method of claim 19, wherein the substance interacting with the physiologically active substance is detected or measured by surface plasmon resonance analysis.
